(19) United States
(12) Patent Application Publication
(10) Pub. No.: US 2026/0248781 A1
(43) Pub. Date: Aug. 27, 2026

(54) SOLUBLE GUANYLATE CYCLASE ACTIVATORS FOR USE IN THE TREATMENT OF HEART FAILURE WITH PRESERVED EJECTION FRACTION IN WOMEN

(71) Applicant: BAYER AKTIENGESELLSCHAFT, Leverkusen (DE)

(72) Inventors: Christiane OTTO, Wuppertal (DE); Ilka MATHAR, Düsseldorf (DE); Michael BECKA, Ense (DE); Peter SANDNER, Wuppertal (DE)

(21) Appl. No.: 18/872,407

(22) PCT Filed: Jun. 7, 2023
(86) PCT No.: PCT/EP2023/065171

§ 371 Date: Dec. 6, 2024

(30) Foreign Application Priority Data
Jun. 9, 2022 (EP) 22178111.5

(51) Int. Cl. A61K 31/496; A61K 31/196; A61P 9/04

(52) U.S. Cl. CPC A61K 31/496; A61K 31/196; A61P 9/04

(57) ABSTRACT

The invention relates to soluble guanylate cyclase (sGC) activators, preferably (3S)-3-(4-chloro-3-{[(2S,3R)-2-(4-chlorophenyl)-4,4,4-trifluoro-3-methylbutanoyl]amino}phenyl)-3-cyclopropylpropanoic acid (runcaciguat) and/or substituted pyrazolo piperidine carboxylic acids, preferably selected from the list consisting of 1-[1-{4-Chloro-4′-[4-(2-methylpropyl)piperazin-1-yl][1,1′-biphenyl]-2-yl}piperidin-3-yl]-5-(difluoromethyl)-1H-pyrazole-4-carboxylic acid, 1-{3(R)-1-[4-Chloro-4′-(4-isobutylpiperazin-1yl)[biphenyl]-2-yl]piperidin-3-yl}-5-(difluoromethyl)-1H-pyrazole-4-carboxylic acid, 1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride, 1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride hemihydrate or 1-[1-{4-chloro-4′-[4-(2-methylpropyl)piperazin-1-yl][1,1′-biphenyl]-2-yl}piperidin-3-yl]-5-(trifluoro-methyl)-1H-pyrazole-4-carboxylic acid hydrochloride (Enantiomer 1) for use in the treatment and/or prophylaxis of heart failure with preserved ejection fraction (HFpEF) in women and their use for preparing medicaments for the use in the treatment and/or prophylaxis of heart failure with preserved ejection fraction (HFpEF) in women.

The invention relates to soluble guanylate cyclase (sGC) activators, preferably (3S)-3-(4-chloro-3-{[(2S,3R)-2-(4-chlorophenyl)-4,4,4-trifluoro-3-methylbutanoyl]amino}phenyl)-3-cyclopropylpropanoic acid (runcaciguat) and/or substituted pyrazolo piperidine carboxylic acids, preferably selected from the list consisting of 1-[1-{4-Chloro-4′-[4-(2-methylpropyl)piperazin-1-yl][1,1′-biphenyl]-2-yl}piperidin-3-yl]-5-(difluoromethyl)-1H-pyrazole-4-carboxylic acid, 1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid, 1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride, 1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride hemihydrate or 1-[1-{4-chloro-4′-[4-(2-methylpropyl)piperazin-1-yl][1,1′-biphenyl]-2-yl}piperidin-3-yl]-5-(trifluoro-methyl)-1H-pyrazole-4-carboxylic acid hydrochloride (Enantiomer 1) for use in the treatment and/or prophylaxis of heart failure with preserved ejection fraction (HFpEF) in women and their use for preparing medicaments for the use in the treatment and/or prophylaxis of heart failure with preserved ejection fraction (HFpEF) in women.

Suitable sGC activators are known from the following publications (the subject-matter disclosed in the publications below hereby also forms part of the subject-matter of the disclosure of the present application): WO2013/157528, WO2015/056663, WO2009/123316, WO2016/001875, WO2016/001876, WO2016/001878, WO2000/02851, WO2012/122340, WO2013/025425, WO2014/039434, WO2016/014463, WO2009/068652, WO2009/071504, WO2010/015652, WO2010/015653, WO2015/033307, WO2016/042536, WO2009/032249, WO2010/099054, WO2012/058132, US2010/0216764, WO01/19776, WO01/19780, WO01/19778, WO02/070459, WO02/070460, WO02/070510, WO02/070462, WO2007/045366, WO2007/045369, WO2007/045433, WO2007/045370, WO2007/045367, WO2014/012935, WO2014/012934, WO2011/141409, WO2008/119457, WO2008/119458, WO2009/127338, WO2010/102717, WO2011/051165, WO2012/076466, WO2012/139888, WO2013/174736.

The following sGC activators are of particular importance:

(3S)-3-(4-chloro-3-{[(2S,3R)-2-(4-chlorophenyl)-4,4,4-trifluoro-3-methylbutanoyl]amino}phenyl)-3-cyclopropylpropanoic acid (runcaciguat) of the formula (I)

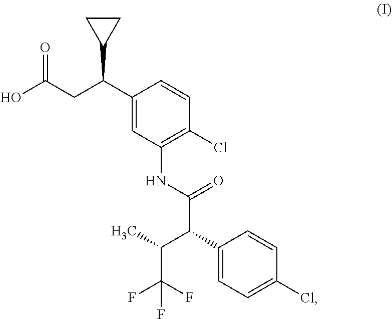 [SMILES: C[C@H]([C@H](C(=O)Nc1cc([C@@H](CC(=O)O)C2CC2)ccc1Cl)c1ccc(Cl)cc1)C(F)(F)F]

which is disclosed in WO2012/139888.

1-[1-{4-Chloro-4′-[4-(2-methylpropyl)piperazin-1-yl][1,1′-biphenyl]-2-yl}piperidin-3-yl]-5-(difluoromethyl)-1H-pyrazole-4-carboxylic acid of the formula (II)

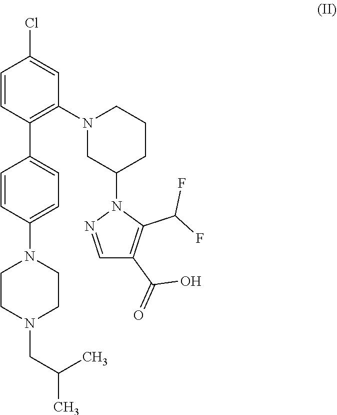 [SMILES: CC(C)CN1CCN(c2ccc(-c3ccc(Cl)cc3N3CCCC(n4ncc(C(=O)O)c4C(F)F)C3)cc2)CC1]

which is disclosed in WO2022/122910.

1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid of the formula (III)

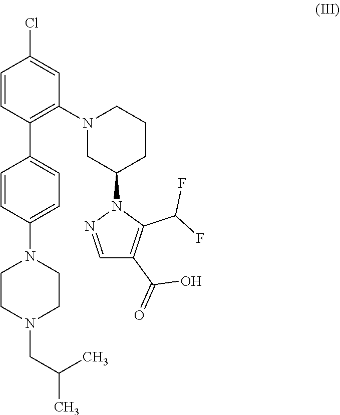 [SMILES: CC(C)CN1CCN(c2ccc(-c3ccc(Cl)cc3N3CCC[C@@H](n4ncc(C(=O)O)c4C(F)F)C3)cc2)CC1]

which is disclosed in WO2022/122910.

1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride of the formula (IV)

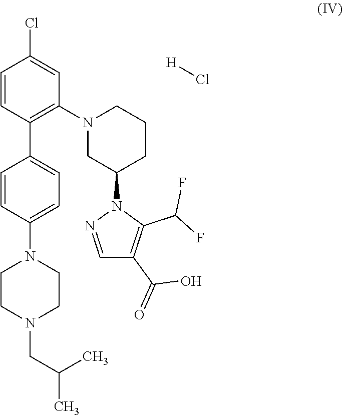 [SMILES: CC(C)CN1CCN(c2ccc(-c3ccc(Cl)cc3N3CCC[C@@H](n4ncc(C(=O)O)c4C(F)F)C3)cc2)CC1 ; Cl]

which is disclosed in WO2022/122910.

1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride hemihydrate of the formula (V)

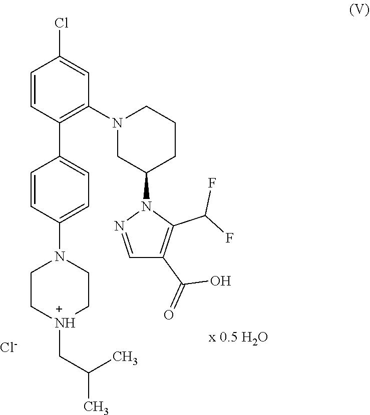 [SMILES: [Cl-] ; CC(C)C[NH+]1CCN(c2ccc(-c3ccc(Cl)cc3N3CCC[C@@H](n4ncc(C(=O)O)c4C(F)F)C3)cc2)CC1]

which is disclosed in WO2022/122910.

1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylic acid hydrochloride (Enantiomer 1) of the formula (VI)

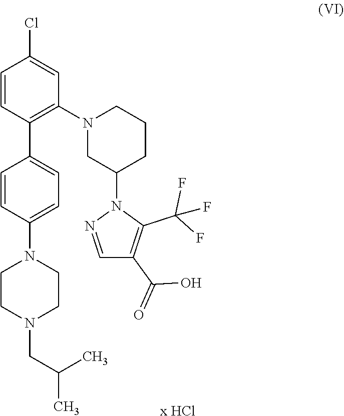 [SMILES: CC(C)CN1CCN(c2ccc(-c3ccc(Cl)cc3N3CCCC(n4ncc(C(=O)O)c4C(F)(F)F)C3)cc2)CC1 ; "x HCl"]

which is disclosed in WO2022/122914.

One of the most important cellular signalling systems in mammalian cells is the cyclic guanosine monophosphate (cGMP) signal transduction. Together with nitric oxide (NO), which is released from the endothelium and transmits hormonal and mechanical signals, it forms the NO/cGMP system. Guanylate cyclases catalyse the biosynthesis of cGMP from guanosine triphosphate (GTP). The representatives of this family disclosed to date can be divided both according to structural features and according to the type of ligands into two groups: the particulate guanylate cyclases which can be stimulated by natriuretic peptides, and the soluble guanylate cyclases which can be stimulated by NO. The soluble guanylate cyclases consist of two subunits which contain one haem per heterodimer, which is part of the regulatory site. The haem domain (H-NOX) is a prerequisite for sGC activation. NO is able to bind to the iron atom of haem and thus markedly increase the activity of the enzyme. Haem-free preparations cannot be stimulated by NO.

Through the production of cGMP and the regulation, resulting therefrom, of phosphodiesterases, ion channels and protein kinases, guanylate cyclase plays a crucial part in various physiological processes, in particular in the relaxation of smooth muscle cells, in platelet aggregation and adhesion and in neuronal signal transmission, but also in fibrotic remodelling and inflammation. Under pathophysiological conditions, the NO/cGMP system may be suppressed, which may lead for example to high blood pressure, platelet activation, increased cellular proliferation and fibrosis, endothelial dysfunction, atherosclerosis, angina pectoris, heart failure, thrombosis, stroke and myocardial infarction or chronic kidney disease

A possible way of treating such disorders which is independent of NO and aims at influencing the cGMP signaling pathway in organisms is a promising approach because of the high efficiency and few side effects which are to be expected.

Compounds, such as organic nitrates, whose effect is based on NO have to date been exclusively used for the therapeutic stimulation of soluble guanylate cyclase. NO is produced by bioconversion and activates is soluble guanylate cyclase by attaching to the central iron atom of haem. Besides the side effects, the development of tolerance is one of the crucial disadvantages of this mode of treatment [O. V. Evgenov et al., Nature Rev. Drug Disc. 5 (2006), 755].

Substances which directly stimulate soluble guanylate cyclase, i.e. without previous release of NO, have been identified in recent years and riociguat and vericiguat have been approved in 2013 and 2021 [Evgenov et al., ibid., Sandner P, Zimmer DP, Milne GT, Follmann M, Hobbs A, Stasch JP. Soluble Guanylate Cyclase Stimulators and Activators. Handb Exp Pharmacol. 2021; 264:355-394.

A common characteristic of this substance class of sGC stimulators is a NO-independent and selective activation of the haem-containing sGC. In addition, the sGC stimulators in combination with NO have a synergistic effect on sGC activation based on a stabilization of the nitrosyl-haem complex. The exact binding site of the sGC stimulators at the sGC is still being debated, but the haem-free enzyme cannot be stimulated by the sGC stimulators mentioned above [Evgenov et al., ibid., Sandner et al. 2021].

However, NO- and haem-independent sGC activators, with BAY 58-2667 as prototype of this class, have been identified. Common characteristics of these substances are that in combination with NO they only have an additive effect on enzyme activation, and that the activation of the oxidized or haem-free enzyme is markedly higher than that of the haem-containing enzyme [Evgenov et al., ibid.; J. P. Stasch et al., Br. J. Pharmacol. 136 (2002), 773; J. P. Stasch et al., J. Clin. Invest. 116 (2006), 2552, Sandner et al. 2021].

The compounds described in the present invention are now likewise capable of activating the haem-free form of soluble guanylate cyclase. This is also confirmed by the fact that these novel activators firstly have no synergistic action with NO at the haem-containing enzyme and that secondly their action cannot be blocked by the haem-dependent inhibitor of soluble guanylate cyclase, 1H-1,2,4-oxadiazolo[4,3-a]-quinoxalin-1-one (ODQ), but is even potentiated by this inhibitor [cf. O. V. Evgenov et al., Nature Rev. Drug Disc. 5 (2006), 755; J. P. Stasch et al., J. Clin. Invest. 116 (2006), 2552]. The sGC activator Runcaciguat (Hahn et al., Drugs Future 43 (2018), 738, WO 2012/139888) is in clinical development by BAYER (https://www.clinicaltrials.gov/NCT04507061).

Our understanding of the redox equilibrium of the sGC in health and diseases is still very limited. Therefore, the treatment potential of sGC activators is not fully clear yet. However, since oxidative stress could render the sGC enzyme heme-free, the sGC activators might have a broad treatment potential.

Heart failure (HF) affects more than 40 million people worldwide and can be divided into two main subgroups: HF with reduced ejection fraction (HFrEF) and HF with preserved ejection fraction (HFpEF). Reduced pumping function of the heart is the main symptom of HFrEF whereas HFpEF (with a left-ventricular ejection fraction≥50%) is characterized by impaired diastolic filling of the heart due to reduced relaxation capability of the diseased myocardium (A. R. Sabbatini, G. Kararigas, J. Am. Coll. Cardiol. 2020; 75(9):1074-1082). Characteristic pathophysiological alterations seen in HFpEF are left-ventricular hypertrophy and cardiac fibrosis leading to impaired diastolic relaxation and increased left-ventricular filling pressures (M. R. Zile, C. F. Baicu. J. Cardiovasc. Transl. Res. 2013; 6 (4):501-515), increased cardiomyocyte stiffness, and systemic inflammation (S. J. Shah et al., Circulation 2016; 134 (1):73-90). Comorbidities such as obesity, renal impairment, diabetes, and hypertension are highly prevalent in HFpEF (A. R. Sabbatini, G. Kararigas, J. Am. Coll. Cardiol. 2020; 75(9):1074-1082, for review). HFpEF has an annual mortality rate between 10 to 30% (C. W. Yancy et al., J. Am. Coll. Cardiol. 2013; 62 (16):1495-1539). Approximately two thirds of patients suffering from HFpEF are women (D. C. Scantlebury, B. A. Borlaug, Curr. Opin. Cardiol. 2011; 26 (6):562-568). As postmenopausal women are predominantly affected by HFpEF, it has been speculated that the decline in estradiol during this period of life could contribute to the development of left-ventricular remodeling, left-ventricular hypertrophy, and diastolic dysfunction in HFpEF. In line with this, it could be demonstrated that left-ventricular hypertrophy in postmenopausal women (J. P. Singh et al., Am. J. Cardiol. 1999; 83 (6):1132-1134) as well as diastolic dysfunction could be improved by postmenopausal hormone therapy. Several pathomechanisms which contribute to HFpEF development such as enhanced oxidative stress, endothelial dysfunction, arterial hypertension, inflammation, obesity, increased extracellular matrix deposition and reduced levels of atrial natriuretic and brain peptide are inhibited by estradiol in premenopausal women and are activated during menopause (when estradiol declines), which might explain why women are more prone to HFpEF development compared to men (A. R. Sabbatini, G. Kararigas, J. Am. Coll. Cardiol. 2020; 75(9):1074-1082, for review).

While a variety of drugs with different mode of actions have been approved for the treatment of HFrEF, many outcome trials in HFpEF failed and only very recently the first two therapeutics were approved for HFpEF: Entresto (a combination of the angiotensin receptor blocker valsartan with neprilysin) and sodium-glucose linked transporter 2 inhibitors (SGLT2 inhibitors). The outcome trial for Entresto in HFpEF was overall neutral (S. D. Solomon et al., PARAGON, New. Engl. J. Med. 2019; 381 (17):1609-1620). In the post hoc analysis, there was a statistically significant relative risk reduction in the primary composite endpoint in two subpopulations: in women and in patients with a left-ventricular ejection fraction≤57%. Based on the combined post hoc analysis of the two outcome trials—one performed in HFrEF (J. J. V. McMurray et al., PARADIGM, New. Engl. J. Med. 2014; 371(11):993-1004) and one performed in HFpEF (S. D. Solomon et al., New. Engl. J. Med. 2019; 381:1609-1620)—the therapeutic efficacy of Entresto within a certain range of left-ventricular ejection fraction was considered ‘biologically plausible’ (S. D. Solomon et al., New. Engl. J. Med. 2019; 381:1609-1620), while the observed sex-specific treatment effect in PARAGON was neglected. However, these data might suggest that female HfpEF patients could respond differently to chronic heart failure treatments. Since women are predominantly affected and considering the high disease burden and mortality in HfpEF patients, there is still a high medical need for new drugs treating this disease, especially in female patients. The underlying problem of the present invention lies in the provision of new medications for the treatment of women suffering from HfpEF.

The sGC activator (3S)-3-(4-chloro-3-{[(2S,3R)-2-(4-chlorophenyl)-4,4,4-trifluoro-3-methylbutanoyl]amino}phenyl)-3-cyclopropylpropanoic acid (runcaciguat) of the formula (I)

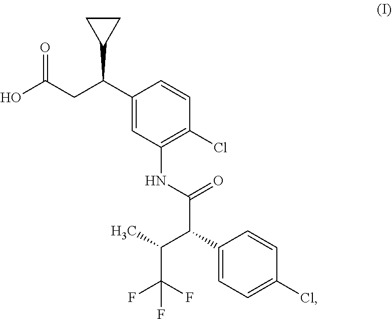 [SMILES: C[C@H]([C@H](C(=O)Nc1cc([C@@H](CC(=O)O)C2CC2)ccc1Cl)c1ccc(Cl)cc1)C(F)(F)F]

as well as its pharmaceutical activity is disclosed in WO2012/139888. However, it is not known that this molecule would be a suitable medicament for use in the treatment of heart failure with preserved ejection fraction (HfpEF) in women.

It is therefore an object of the present invention to provide novel medicaments for use in the treatment of heart failure with preserved ejection fraction (HfpEF) in women comprising a sGC activator.

Surprisingly, it has now been found that certain substituted pyrazolo piperidine carboxylic acids as well as their corresponding salts represent highly potent sGC activators with good pharmacokinetic behavior with a good pharmacological activity profile as well as beneficial physico-chemical properties (e.g. solubility). Furthermore it has been found that the sGC activator 3S)-3-(4-chloro-3-{[(2S,3R)-2-(4-chlorophenyl)-4,4,4-trifluoro-3-methylbutanoyl]amino}phenyl)-3-cyclopropylpropanoic acid (runcaciguat) as well as certain substituted pyrazolo piperidine carboxylic acids selected form the list consisting of 1-[1-{4-Chloro-4′-[4-(2-methylpropyl)piperazin-1-yl][1,1′-biphenyl]-2-yl}piperidin-3-yl]-5-(difluoromethyl)-1H-pyrazole-4-carboxylic acid, 1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid, 1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride, 1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride hemihydrate or 1-[1-{4-chloro-4′-[4-(2-methylpropyl)piperazin-1-yl][1,1′-biphenyl]-2-yl}piperidin-3-yl]-5-(trifluoro-methyl)-1H-pyrazole-4-carboxylic acid hydrochloride (Enantiomer 1) are suitable as novel medicaments for use in the treatment of heart failure with preserved ejection fraction (HFpEF) in women.

Accordingly, the invention provides a compound for use in the treatment and/or prophylaxis of heart failure with preserved ejection fraction (HFpEF) in women selected from the group consisting of

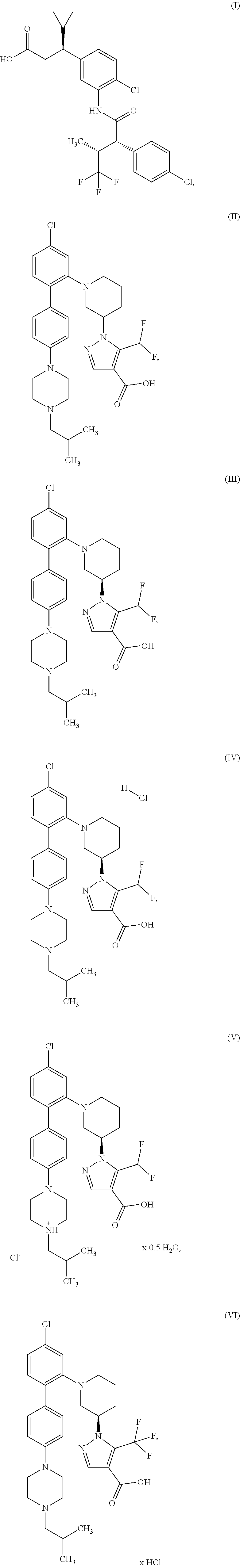

or one of the salts thereof, solvates thereof or solvates of the salts thereof.

Accordingly, the invention provides a compound for use in the treatment and/or prophylaxis of heart failure with preserved ejection fraction (HFpEF) in women selected from the group consisting of

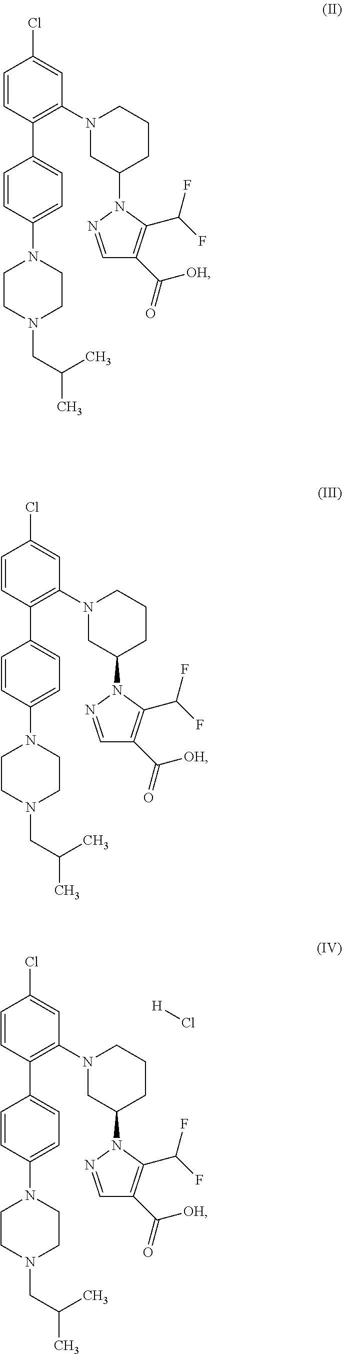 [SMILES: CC(C)CN1CCN(c2ccc(-c3ccc(Cl)cc3N3CCCC(n4ncc(C(=O)O)c4C(F)F)C3)cc2)CC1 ; CC(C)CN1CCN(c2ccc(-c3ccc(Cl)cc3N3CCC[C@@H](n4ncc(C(=O)O)c4C(F)F)C3)cc2)CC1 ; CC(C)CN1CCN(c2ccc(-c3ccc(Cl)cc3N3CCC[C@@H](n4ncc(C(=O)O)c4C(F)F)C3)cc2)CC1 ; Cl]

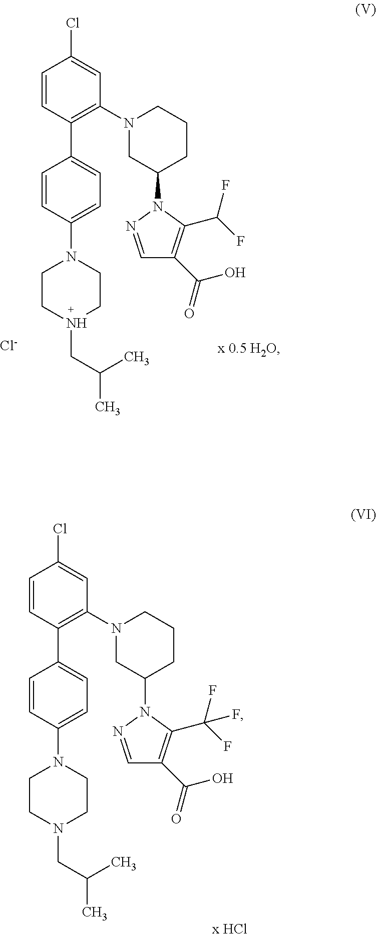 [SMILES: [Cl-] ; CC(C)CN1CCN(c2ccc(-c3ccc(Cl)cc3N3CCC[C@@H](n4ncc(C(=O)O)c4C(F)F)C3)cc2)CC1 ; CC(C)CN1CCN(c2ccc(-c3ccc(Cl)cc3N3CCCC(n4ncc(C(=O)O)c4C(F)(F)F)C3)cc2)CC1]

or one of the salts thereof, solvates thereof or solvates of the salts thereof.

Preferred salts in the context of the present invention are physiologically acceptable salts of the compounds according to the invention. However, the invention also encompasses salts which themselves are unsuitable for pharmaceutical applications, but which can be used, for example, for the isolation or purification of the compounds according to the invention.

Physiologically acceptable salts of the compounds according to the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

The present invention includes all possible salts of the compounds according to the invention as single salts, or as any mixture of said salts, in any ratio.

Solvates in the context of the invention are described as those forms of the inventive compounds which form a complex in the solid or liquid state by coordination with solvent molecules. The compounds according to the invention may contain polar solvents, in particular water, methanol or ethanol for example, as structural element of the crystal lattice of the compounds. Hydrates are a specific form of the solvates in which the coordination is with water. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

The compounds of the invention have valuable pharmacological properties and can be used for prevention and treatment of diseases in humans and animals.

The compounds according to the invention are potent activators of soluble guanylate cyclase. They lead to vasorelaxation, inhibition of platelet aggregation and lowering of blood pressure and increase of coronary and renal blood flow. These effects are mediated via direct heme-independent activation of soluble guanylate cyclase and an increase of intracellular cGMP.

In addition, the compounds according to the invention have advantageous pharmacokinetic properties, in particular with respect to their bioavailability and/or duration of action after intravenous or oral administration.

The compounds according to the invention have an unforeseeable useful pharmacological activity spectrum and good pharmacokinetic behavior, in particular a sufficient exposure of such a compound in the blood above the minimal effective concentration within a given dosing interval after oral administration. Such a profile results in an improved peak-to-trough ratio (quotient of maximum to minimum concentration) within a given dosing interval, which has the advantage that the compound can be administered less frequently and at a significantly lower dose to achieve an effect. They are compounds that activate soluble guanylate cyclase.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

In the context of the present invention, the terms “prevention”, “prophylaxis” and “preclusion” are used synonymously and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

The compounds according to the invention are particularly suitable for the treatment and/or prevention of cardiovascular and cardiac diseases, preferably heart failure with preserved ejection fraction (HFpEF) in women.

Accordingly, the compounds according to the invention can be used in medicaments for the treatment and/or prevention of heart failure, preferably heart failure with preserved ejection fraction (HFpEF) in women.

In the context of the present invention, the term “heart failure” encompasses both acute and chronic forms of heart failure, and also more specific or related types of disease, such as acute decompensated heart failure, right heart failure, left heart failure, global heart failure, also diastolic heart failure and systolic heart failure, heart failure with reduced ejection fraction (HFrEF), heart failure with preserved ejection fraction (HFpEF), heart failure with mid-range ejection fraction (HFmEF), ischemic cardiomyopathy, dilated cardiomyopathy, hypertrophic cardiomyopathy, idiopathic cardiomyopathy, congenital heart defects and cardiomyopathies, heart valve defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, and also diastolic heart failure and systolic heart failure, heart failure with reduced ejection fraction (HFrEF), heart failure with preserved ejection fraction (HFpEF).

We tested sGC activators, e.g. runcaciguat and substituted pyrazolo piperidine carboxylic acids, preferably selected from the list consisting of 1-[1-{4-Chloro-4′-[4-(2-methylpropyl)piperazin-1-yl][1,1′-biphenyl]-2-yl}piperidin-3-yl]-5-(difluoromethyl)-1H-pyrazole-4-carboxylic acid, 1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid, 1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride, 1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride hemihydrate or 1-[1-{4-chloro-4′-[4-(2-methylpropyl)piperazin-1-yl][1,1′-biphenyl]-2-yl}piperidin-3-yl]-5-(trifluoro-methyl)-1H-pyrazole-4-carboxylic acid hydrochloride (Enantiomer 1), in a rat model of HFpEF which is characterized by hypertension, endothelial dysfunction and oxidative stress. These rats present with increased heart and kidney damage resulting in a high mortality. For this model, historically male rats are used as also in many clinical trials the majority of patients is male. We could show that sGC activation by runcaciguat (example 1) and also by 1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid (example 3) had a dose-dependent, beneficial effect on both survival and kidney function which are selected for this invention as very predictive read-outs for the HFpEF outcome.

We have surprisingly shown gender-specific effects in the treatment of HFpEF. Providing an effective treatment option for female HFpEF patients.

The present invention furthermore provides the use of the compounds according to the invention for the treatment and/or prevention of heart failure with preserved ejection fraction (HFpEF) in women.

The present invention furthermore provides the use of the compounds according to the invention for preparing a medicament for the treatment and/or prevention of heart failure with preserved ejection fraction (HFpEF) in women.

The present invention furthermore provides a medicament comprising at least one of the compounds according to the invention for the treatment and/or prevention of heart failure with preserved ejection fraction (HFpEF) in women.

The present invention furthermore provides the use of the compounds according to the invention in a method for the treatment and/or prevention of heart failure with preserved ejection fraction (HFpEF) in women.

The present invention furthermore provides a method for the treatment and/or prevention of heart failure with preserved ejection fraction (HFpEF) in women, using an effective amount of at least one of the compounds according to the invention.

The present invention further provides medicaments comprising a compound according to the invention and one or more further active compounds.

The compounds of the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Suitable for extraocular (topic) administration are administration forms which operate in accordance with the prior art, which release the active compound rapidly and/or in a modified or controlled manner and which contain the active compound in crystalline and/or amorphized and/or dissolved form such as, for example, eye drops, sprays and lotions (e.g. solutions, suspensions, vesicular/colloidal systems, emulsions, aerosols), powders for eye drops, sprays and lotions (e.g. ground active compound, mixtures, lyophilisates, precipitated active compound), semisolid eye preparations (e.g. hydrogels, in-situ hydrogels, creams and ointments), eye inserts (solid and semisolid preparations, e.g. bioadhesives, films/wafers, tablets, contact lenses).

Preference is given to oral administration.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixture agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprises at least one compound according to the invention, conventionally together with one or more pharmaceutically suitable excipient(s), and to their use according to the present invention.

An embodiment of the invention are pharmaceutical compositions comprising at least one compound of formula (I) according to the invention, preferably together with at least one inert, non-toxic, pharmaceutically suitable auxiliary, and the use of these pharmaceutical compositions for the above cited purposes.

In accordance with another aspect, the present invention covers pharmaceutical combinations, in particular medicaments, comprising at least one compound of general formula (I) of the present invention and at least one or more further active ingredients, in particular for the treatment and/or prophylaxis of cardiovascular disorders, preferably heart failure with preserved ejection fraction (HFpEF) in women.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The inventive compounds can be employed alone or, if required, in combination with other active ingredients. The present invention further provides medicaments comprising at least one of the inventive compounds and one or more further active ingredients, especially for treatment and/or prophylaxis of the aforementioned disorders. Preferred examples of suitable active ingredient combinations include:
organic nitrates and NO donors, for example sodium nitroprusside, nitroglycerin, isosorbide mononitrate, isosorbide dinitrate, molsidomine or SIN-1, and inhaled NO;
compounds which inhibit the breakdown of cyclic guanosine monophosphate (cGMP), for example inhibitors of phosphodiesterases (PDE) 1, 2, 5 and/or 9, especially PDE 5 inhibitors such as sildenafil, vardenafil, tadalafil, udenafil, desantafil, avanafil, mirodenafil, lodenafil or PF-00489791;
compounds which inhibit the breakdown of cyclic adenosine monophosphate (cAMP), for example inhibitors of phosphodiesterases (PDE) 3 and 4, especially cilostazole, milrinone, roflumilast, apremilast, or crisaborole;
hypotensive active ingredients, by way of example and with preference from the group of the calcium antagonists, angiotensin AII antagonists, ACE inhibitors, NEP-inhibitors, vasopeptidase-inhibitors, endothelin antagonists, renin inhibitors, alpha-receptor blockers, beta-receptor blockers, mineralocorticoid receptor antagonists, rho-kinase-inhibitors and diuretics;
antiarrhythmic agents, by way of example and with preference from the group of sodium channel blocker, beta-receptor blocker, potassium channel blocker, calcium antagonists, If-channel blocker, digitalis, parasympatholytics (vagolytics), sympathomimetics and other antiarrhythmics as adenosin, adenosine receptor agonists as well as vernakalant;
positive-inotrop agents, by way of example cardiac glycoside (Digoxin), beta-adrenergic and dopaminergic agonists, such as isoprenaline, adrenalin, noradrenalin, dopamin or dobutamin;
vasopressin-receptor-antagonists, by way of example and with preference from the group of conivaptan, tolvaptan, lixivaptan, mozavaptan, satavaptan, pecavaptan, SR-121463, RWJ 676070 or BAY 86-8050, as well as the compounds described in WO 2010/105770, WO2011/104322 and WO 2016/071212;
active ingredients which alter lipid metabolism, for example and with preference from the group of the thyroid receptor agonists, cholesterol synthesis inhibitors such as, by way of example and preferably, HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.
bronchodilatory agents, for example and with preference from the group of the beta-adrenergic receptor-agonists, such as, by way of example and preferably, albuterol, isoproterenol, metaprotereno17aricalcitolin, formoterol or salmeterol, or from the group of the anticholinergics, such as, by way of example and preferably, ipratropiumbromid;
anti-inflammatory agents, for example and with preference from the group of the glucocorticoids, such as, by way of example and preferably, prednison, prednisolon, methylprednisolon, triamcinolon, dexamethason, beclomethason, betamethason, flunisolid, budesonid or fluticason as well as the non-steroidal anti-inflammatory agents (NSAIDs), by way of example and preferably, acetyl salicylic acid (aspirin), ibuprofen and naproxen, 5-amino salicylic acid-derivates, leukotriene-antagonists, TNF-alpha-inhibitors and chemokine-receptor antagonists, such as CCR1, 2 and/or 5 inhibitors;
agents modulating the immune system, for example immunoglobulins;
agents that inhibit the signal transductions cascade, for example and with preference from the group of the kinase inhibitors, by way of example and preferably, from the group of the tyrosine kinase- and/or serine/threonine kinase inhibitors;
agents, that inhibit the degradation and modification of the extracellular matrix, for example and with preference from the group of the inhibitors of the matrix-metalloproteases (MMPs), by way of example and preferably, inhibitors of chymasee, stromelysine, collagenases, gelatinases and aggrecanases (with preference from the group of MMP-1, MMP-3, MMP-8, MMP-9, MMP-10, MMP-11 and MMP-13) as well as of the metallo-elastase (MMP-12) and neutrophil-elastase (HNE), as for example sivelestat or DX-890;
agents, that block the binding of serotonin to its receptor, for example and with preference antagonists of the 5-HT2b-receptor;
organic nitrates and NO-donators, for example and with preference sodium nitroprussid, nitro-glycerine, isosorbid mononitrate, isosorbid dinitrate, molsidomine or SIN-1, as well as inhaled NO;
NO-independent, but heme-dependent stimulators of the soluble guanylate cyclase, for example and with preference the compounds described in WO 00/06568, WO 00/06569, WO 02/42301, WO 03/095451, WO 2011/147809, WO 2012/004258, WO 2012/028647 and WO 2012/059549;
NO-independent and heme-independent activators of the soluble guanylate cyclase, for example and with preference the compounds described in WO 01/19355, WO 01/19776, WO 01/19778, WO 01/19780, WO 02/070462 and WO 02/070510 beschriebenen Verbindungen;
agents, that stimulate the synthesis of cGMP, like for example sGC modulators, for example and with preference riociguat, cinaciguat, vericiguat or runcaciguat;
prostacyclin-analogs, for example and with preference iloprost, beraprost, treprostinil or epoprostenol;
agents, that inhibit soluble epoxidhydrolase (sEH), for example and with preference N,N′-Di-cyclohexyl urea, 12-(3-Adamantan-1-yl-ureido)-dodecanic acid or 1-Adamantan-1-yl-3-{5-[2-(2-ethoxyethoxy)ethoxy]pentyl}-urea;
agents that interact with glucose metabolism, for example and with preference insuline, biguanide, thiazolidinedione, sulfonyl urea, acarbose, DPP4 inhibitors, GLP-1 analogs or SGLT-2 inhibitors, for example empagliflozin, dapagliflozin, canagliflozin, sotagliflozin;
natriuretic peptides, for example and with preference atrial natriuretic peptide (ANP), natriuretic peptide type B (BNP, Nesiritid), natriuretic peptide type C (CNP) or urodilatin;
activators of the cardiac myosin, for example and with preference omecamtiv mecarbil (CK-1827452);
calcium-sensitizers, for example and with preference levosimendan;
agents that affect the energy metabolism of the heart, for example and with preference etomoxir, dichloroacetat, ranolazine or trimetazidine, full or partial adenosine A1 receptor agonists such as GS-9667 (formerly known as CVT-3619), capadenoson, neladenoson and neladenoson bialanate;
agents that affect the heart rate, for example and with preference ivabradin;
cyclooxygenase inhibitors such as, for example, bromfenac and nepafenac;
inhibitors of the kallikrein-kinin system such as, for example, safotibant and ecallantide;
inhibitors of the sphingosine 1-phosphate signal paths such as, for example, sonepcizumab;
inhibitors of the complement-C5a receptor such as, for example, eculizumab;
plasminogen activators (thrombolytics/fibrinolytics) and compounds which promote thrombolysis/fibrinolysis such as inhibitors of the plasminogen activator inhibitor (PAI inhibitors) or inhibitors of the thrombin-activated fibrinolysis inhibitor (TAFI inhibitors) such as, for example, tissue plasminogen activator (t-PA, for example Actilyse®), streptokinase, reteplase and urokinase or plasminogen-modulating substances causing increased formation of plasmin;
anticoagulatory substances (anticoagulants) such as, for example, heparin (UFH), low-molecular-weight heparins (LMW), for example tinzaparin, certoparin, pamaparin, nadroparin, ardeparin, enoxaparin, reviparin, dalteparin, danaparoid, semuloparin (AVE 5026), adomiparin (M118) and EP-42675/ORG42675;
direct thrombin inhibitors (DTI) such as, for example, Pradaxa (dabigatran), atecegatran (AZD-0837), DP-4088, SSR-182289A, argatroban, bivalirudin and tanogitran (BIBT-986 and prodrug BIBT-1011) and hirudin;
direct factor Xa inhibitors such as, for example, rivaroxaban, apixaban, edoxaban (DU-176b), betrixaban (PRT-54021), R-1663, darexaban (YM-150), otamixaban (FXV-673/RPR-130673), letaxaban (TAK-442), razaxaban (DPC-906), DX-9065a, LY-517717, tanogitran (BIBT-986, prodrug: BIBT-1011), idraparinux and fondaparinux;
inhibitors of coagulation factor XI and XIa such as, for example, FXI ASO-LICA, fesomersen, BAY 121-3790, MAA868, BMS986177, EP-7041 and AB-022;
substances which inhibit the aggregation of platelets (platelet aggregation inhibitors, thrombocyte aggregation inhibitors), such as, for example, acetylsalicylic acid (such as, for example, aspirin), P2Y12 antagonists such as, for example, ticlopidine (Ticlid), clopidogrel (Plavix), prasugrel, ticagrelor, cangrelor and elinogrel, and PAR-1 antagonists such as, for example, vorapaxar, and PAR-4 antagonists;
platelet adhesion inhibitors such as GPVI and/or GPIb antagonists such as, for example, Revacept or caplacizumab;
fibrinogen receptor antagonists (glycoprotein-IIb/IIIa antagonists) such as, for example, abciximab, eptifibatide, tirofiban, lamifiban, lefradafiban and fradafiban;
recombinant human activated protein C such as, for example, Xigris or recombinant thrombomodulin.

Antithrombotic agents are preferably understood to mean compounds from the group of the platelet aggregation inhibitors, the anticoagulants or the profibrinolytic substances.

In a preferred embodiment of the invention, the inventive compounds are administered in combination with a platelet aggregation inhibitor, by way of example and with preference aspirin, clopidogrel, prasugrel, ticagrelor, is ticlopidin or dipyridamole.

In a preferred embodiment of the invention, the inventive compounds are administered in combination with a thrombin inhibitor, by way of example and with preference ximelagatran, dabigatran, melagatran, bivalirudin or clexane.

In a preferred embodiment of the invention, the inventive compounds are administered in combination with a GPIIb/IIIa antagonist such as, by way of example and with preference, tirofiban or abciximab.

In a preferred embodiment of the invention, the inventive compounds are administered in combination with a factor Xa inhibitor, by way of example and with preference rivaroxaban (BAY 59-7939), DU-176b, apixaban, betrixaban, otamixaban, fidexaban, razaxaban, letaxaban, eribaxaban, fondaparinux, idraparinux, PMD-3112, darexaban (YM-150), KFA-1982, EMD-503982, MCM-17, MLN-1021, DX 9065a, DPC 906, JTV 803, SSR-126512 or SSR-128428.

In a preferred embodiment of the invention, the inventive compounds are administered in combination with a factor XI or factor XIa inhibitor, by way of example and with preference FXI ASO-LICA, fesomersen, BAY 121-3790, MAA868, BMS986177, EP-7041 or AB-022.

In a preferred embodiment of the invention, the inventive compounds are administered in combination with heparin or with a low molecular weight (LMW) heparin derivative.

In a preferred embodiment of the invention, the inventive compounds are administered in combination with a vitamin K antagonist, by way of example and with preference coumarin.

Hypotensive agents are preferably understood to mean compounds from the group of the calcium antagonists, angiotensin AII antagonists, ACE inhibitors, endothelin antagonists, renin inhibitors, alpha-receptor blockers, beta-receptor blockers, mineralocorticoid receptor antagonists, rho-kinase inhibitors and the diuretics.

In a preferred embodiment of the invention, the inventive compounds are administered in combination with a calcium antagonist, by way of example and with preference nifedipine, amlodipine, verapamil or diltiazem.

In a preferred embodiment of the invention, the inventive compounds are administered in combination with an alpha-1-receptor blocker, by way of example and with preference prazosin.

In a preferred embodiment of the invention, the inventive compounds are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the inventive compounds are administered in combination with an angiotensin AII antagonist, by way of example and with preference losartan, candesartan, valsartan, telmisartan or embusartanor a dual angiotensin AII antagonist/neprilysin-inhibitor, by way of example and with preference LCZ696 (valsartan/sacubitril).

In a preferred embodiment of the invention, the inventive compounds are administered in combination with an ACE inhibitor, by way of example and with preference enalapril, captopril, lisinopril, ramipril, delapril, fosinopril, quinopril, perindopril or trandopril.

In a preferred embodiment of the invention, the inventive compounds are administered in combination with an endothelin antagonist, by way of example and with preference bosentan, darusentan, ambrisentan or sitaxsentan.

In a preferred embodiment of the invention, the inventive compounds are administered in combination with a renin inhibitor, by way of example and with preference aliskiren, SPP-600 or SPP-800.

In a preferred embodiment of the invention, the inventive compounds are administered in combination with a mineralocorticoid receptor antagonist, by way of example and with preference spironolactone, AZD9977, finerenone or eplerenone.

In a preferred embodiment of the invention, the inventive compounds are administered in combination with a loop diuretic, for example furosemide, torasemide, bumetanide and piretanide, with potassium-sparing diuretics, for example amiloride and triamterene, with aldosterone antagonists, for example spironolactone, potassium canrenoate and eplerenone, and also thiazide diuretics, for example hydrochlorothiazide, chlorthalidone, xipamide and indapamide.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and the lipoprotein(a) antagonists.

In a preferred embodiment of the invention, the inventive compounds are administered in combination with a CETP inhibitor, by way of example and with preference dalcetrapib, anacetrapib, torcetrapib (CP-529 414), JJT-705 or CETP vaccine (Avant).

In a preferred embodiment of the invention, the inventive compounds are administered in combination with a thyroid receptor agonist, by way of example and with preference D-thyroxine, 3,5,3′-triiodothyronine (T3), is CGS 23425 or axitirome (CGS 26214).

In a preferred embodiment of the invention, the inventive compounds are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the inventive compounds are administered in combination with a squalene synthesis inhibitor, by way of example and with preference BMS-188494 or TAK-475.

In a preferred embodiment of the invention, the inventive compounds are administered in combination with an ACAT inhibitor, by way of example and with preference avasimibe, melinamide, pactimibe, eflucimibe or SMP-797.

In a preferred embodiment of the invention, the inventive compounds are administered in combination with an MTP inhibitor, by way of example and with preference implitapide, BMS-201038, R-103757 or JTT-130.

In a preferred embodiment of the invention, the inventive compounds are administered in combination with a PPAR-gamma agonist, by way of example and with preference pioglitazone or rosiglitazone.

In a preferred embodiment of the invention, the inventive compounds are administered in combination with a PPAR-delta agonist, by way of example and with preference GW 501516 or BAY 68-5042.

In a preferred embodiment of the invention, the inventive compounds are administered in combination with a cholesterol absorption inhibitor, by way of example and with preference ezetimibe, tiqueside or pamaqueside.

In a preferred embodiment of the invention, the inventive compounds are administered in combination with a lipase inhibitor, a preferred example being orlistat.

In a preferred embodiment of the invention, the inventive compounds are administered in combination with a polymeric bile acid adsorbent, by way of example and with preference cholestyramine, colestipol, colesolvam, CholestaGel or colestimide.

In a preferred embodiment of the invention, the inventive compounds are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the inventive compounds are administered in combination with a lipoprotein(a) antagonist, by way of example and with preference, gemcabene calcium (CI-1027) or nicotinic acid.

In a preferred embodiment of the invention, the inventive compounds are administered in combination with a lipoprotein(a) antagonist, by way of example and with preference, gemcabene calcium (CI-1027) or nicotinic acid.

In a preferred embodiment of the invention, the inventive compounds are administered in combination with sGC modulators, by way of example and with preference, riociguat, cinaciguat or vericiguat.

In a preferred embodiment of the invention, the inventive compounds are administered in combination with an agent affecting the glucose metabolism, by way of example and with preference, insulin, a sulfonyl urea, acarbose, DPP4 inhibitors, GLP-1 analogues or SGLT-1 inhibitors empagliflozin, dapagliflozin, canagliflozin, sotagliflozin.

In a preferred embodiment of the invention, the compounds according to the invention are administered in combination with a TGFbeta antagonist, by way of example and with preference pirfenidone or fresolimumab.

In a preferred embodiment of the invention, the compounds according to the invention are administered in combination with a CCR2 antagonist, by way of example and with preference CCX-140.

In a preferred embodiment of the invention, the compounds according to the invention are administered in combination with a TNFalpha antagonist, by way of example and with preference adalimumab.

In a preferred embodiment of the invention, the compounds according to the invention are administered in combination with a galectin-3 inhibitor, by way of example and with preference GCS-100.

In a preferred embodiment of the invention, the compounds according to the invention are administered in combination with a Nrf-2 inhibitor, by way of example and with preference bardoxolone In a preferred embodiment of the invention, the compounds according to the invention are administered in combination with a BMP-7 agonist, by way of example and with preference THR-184.

In a preferred embodiment of the invention, the compounds according to the invention are administered in combination with a NOX1/4 inhibitor, by way of example and with preference GKT-137831.

In a preferred embodiment of the invention, the compounds according to the invention are administered in combination with a medicament which affects the vitamin D metabolism, by way of example and with preference calcitriol, alfacalcidol, doxercalciferol, maxacalcitol, paricalcitol, cholecalciferol or paracalcitol.

In a preferred embodiment of the invention, the compounds according to the invention are administered in combination with a cytostatic agent, by way of example and with preference cyclophosphamide.

In a preferred embodiment of the invention, the compounds according to the invention are administered in combination with an immunosuppressive agent, by way of example and with preference ciclosporin.

In a preferred embodiment of the invention, the compounds according to the invention are administered in is combination with a phosphate binder, by way of example and with preference colestilan, sevelamer hydrochloride and sevelamer carbonate, lanthanum and lanthanum carbonate.

In a preferred embodiment of the invention, the compounds according to the invention are administered in combination with renal proximal tubule sodium-phosphate co-transporter, by way of example and with preference, niacin or nicotinamide.

In a preferred embodiment of the invention, the compounds according to the invention are administered in combination with a calcimimetic for therapy of hyperparathyroidism.

In a preferred embodiment of the invention, the compounds according to the invention are administered in combination with agents for iron deficit therapy, by way of example and with preference iron products.

In a preferred embodiment of the invention, the compounds according to the invention are administered in combination with agents for the therapy of hyperurikaemia, by way of example and with preference allopurinol or rasburicase.

In a preferred embodiment of the invention, the compounds according to the invention are administered in combination with glycoprotein hormone for the therapy of anemia, by way of example and with preference erythropoietin, daprodustat, molidustat, roxadustat, vadadustat, desidustat.

In a preferred embodiment of the invention, the compounds according to the invention are administered in combination with biologics for immune therapy, by way of example and with preference abatacept, rituximab, eculizumab or belimumab.

In a preferred embodiment of the invention, the compounds according to the invention are administered in combination with vasopressin antagonists (group of the vaptanes) for the treatment of heart failure, by way of example and with preference tolvaptan, conivaptan, lixivaptan, mozavaptan, satavaptan, pecavaptan or relcovaptan.

In a preferred embodiment of the invention, the compounds according to the invention are administered in combination with Jak inhibitors, by way of example and with preference ruxolitinib, tofacitinib, baricitinib, CYT387, GSK2586184, lestaurtinib, pacritinib (SB1518) or TG101348.

In a preferred embodiment of the invention, the compounds according to the invention are administered in combination with prostacyclin analogs for therapy of microthrombi.

In a preferred embodiment of the invention, the compounds according to the invention are administered in combination with an alkali therapy, by way of example and with preference sodium bicarbonate.

In a preferred embodiment of the invention, the compounds according to the invention are administered in combination with an mTOR inhibitor, by way of example and with preference everolimus or rapamycin.

In a preferred embodiment of the invention, the compounds according to the invention are administered in combination with an NHE3 inhibitor, by way of example and with preference AZD1722 or tenapanor.

In a preferred embodiment of the invention, the compounds according to the invention are administered in combination with an eNOS modulator, by way of example and with preference sapropterin.

In a preferred embodiment of the invention, the compounds according to the invention are administered in combination with a CTGF inhibitor, by way of example and with preference FG-3019.

The present invention further provides medicaments which comprise at least one compound according to the invention, typically together with one or more inert, nontoxic, pharmaceutically suitable auxiliaries, and the use thereof for the aforementioned purposes.

The compounds according to the invention may act systemically and/or locally. For this purpose, they can be administered in a suitable manner, for example by the oral, parenteral, pulmonal, nasal, sublingual, lingual, buccal, rectal, dermal, transdermal, conjunctival, otic route, or as an implant or stent.

The compounds according to the invention can be administered in administration forms suitable for these administration routes.

Suitable administration forms for oral administration are those which work according to the prior art, which release the compounds according to the invention rapidly and/or in a modified manner and which contain the compounds according to the invention in crystalline and/or amorphized and/or dissolved form, for example tablets (uncoated or coated tablets, for example with gastric juice-resistant or retarded-dissolution or insoluble coatings which control the release of the compound according to the invention), tablets or films/wafers which disintegrate rapidly in the oral cavity, films/lyophilizates or capsules (for example hard or soft gelatine capsules), sugar-coated tablets, granules, pellets, powders, emulsions, suspensions, aerosols or solutions.

Parenteral administration can bypass an absorption step (e.g. intravenously, intraarterially, intracardially, intraspinally or intralumbally) or include an absorption (e.g. intramuscularly, subcutaneously, intracutaneously, percutaneously or intraperitoneally). Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers), nasal drops, solutions or sprays, tablets, films/wafers or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Oral or parenteral administration is preferred, especially oral and intravenous administration.

The compounds according to the invention can be converted to the administration forms mentioned. This can be done in a manner known per se, by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), dyes (e.g. inorganic pigments, for example iron oxides) and flavour and/or odour correctants.

In general, it has been found to be advantageous in the case of parenteral administration to administer amounts of about 0.001 to 1 mg/kg, preferably about 0.01 to 0.5 mg/kg, of body weight to achieve effective results.

In the case of oral administration, the dosage is about 0.01 to 100 mg/kg, preferably about 0.01 to 20 mg/kg and more preferably about 0.01 to 10 mg/kg, more preferably about 0.01 to 3 mg/kg, also more preferably about 0.03 to 2 mg/kg, also more preferably about 0.03 to 0.7 mg/kg, also more preferably about 0.3 to 2 mg/kg of body weight.

In the case of oral administration, the oral administration/dosage form contains 0.1 mg to 500 mg, preferably 1 mg to 140 mg, more preferably 2.5 mg to 120 mg, also more preferably 20 mg to 120 mg, also more preferably 30 mg to 120 mg, also more preferably 2.5 mg to 50 mg, more preferably 2 to 50 mg, more preferably 2 to 40 mg, more preferably 2 o 30 mg, more preferably 2.5 to 40 mg, more preferably 2.5 to 30 mg. Suitable amounts of active ingredient are for example 1 mg, 2 mg, 2.5 mg, 3 mg, 4 mg, 5 mg, 6 mg, 7 mg, 7.5 mg, 8 mg, 9 mg, 10 mg, 12.5 mg, 15 mg, 17.5. mg, 20 mg, 25 mg, 30 mg, 35 mg, 40 mg, 45 mg, 50 mg, 60 mg, 70 mg, 75 mg, 80 m, 90 mg, 100 mg, 120 mg, 125 mg, 150 mg, 175 mg, or 200 mg.

For compound of formula (I) suitable amounts for oral dosage forms are for example 0.1 mg to 500 mg, preferably 1 mg to 140 mg, preferably 1 mg to 200 mg, more preferably 2.5 mg to 120 mg, also more preferably 20 mg to 120 mg, also more preferably 30 mg to 120 mg. Suitable amounts of active ingredient of formula (I) are for example 25 mg, 30 mg, 35 mg, 40 mg, 45 mg, 50 mg, 60 mg, 70 mg, 75 mg, 80 mg, 90 mg, 100 mg, 120 mg, 125 mg or 130 mg.

For compounds of formula (II), (II), (IV), (V) or (VI) suitable amounts for oral dosage forms are for example 0.1 mg to 500 mg, preferably 1 mg to 140 mg, more preferably 2.5 mg to 50 mg, more preferably 2 mg to 50 mg, more preferably 2 to 40 mg, more preferably 2 mg to 30 mg, more preferably 2.5 mg to 40 mg, more preferably 2.5 mg to 30 mg. Suitable amounts of active ingredient of formula (II), (II), (IV), (V) or (VI) are for example 1 mg, 2 mg, 2.5 mg, 3 mg, 4 mg, 5 mg, 6 mg, 7 mg, 7.5 mg, 8 mg, 9 mg, 10 mg, 12.5 mg, 15 mg, 17.5. mg, 20 mg, 25 mg, 30 mg, 35 mg, 40 mg.

It may nevertheless be necessary where appropriate to deviate from the stated amounts, specifically as a function of the body weight, route of administration, individual response to the active compound, nature of the preparation and time or interval over which administration takes place. For instance, in some cases, less than the aforementioned minimum amount may be sufficient, while in other cases the upper limit mentioned must be exceeded. In the case of administration of relatively large amounts, it may be advisable to divide these into several individual doses over the course of the day.

The total amount of the active ingredient to be administered will generally range from about 0.001 mg/kg to about 200 mg/kg body weight per day, and preferably about 0.01 to 100 mg/kg, preferably about 0.01 to 20 mg/kg and more preferably about 0.01 to 10 mg/kg, more preferably about 0.01 to 3 mg/kg, also more preferably about 0.03 to 2 mg/kg, also more preferably about 0.03 to 0.7 mg/kg, also more preferably about 0.3 to 2 mg/kg of body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient and to be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course, the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Nevertheless, it may optionally be necessary to deviate from the stated amounts, namely depending on body is weight, route of administration, individual response to the active substance, type of preparation and time point or interval when application takes place. Thus, in some cases it may be sufficient to use less than the aforementioned minimum amount, whereas in other cases the stated upper limit must be exceeded. When applying larger amounts, it may be advisable to distribute these in several individual doses throughout the day.

According to a further embodiment, the compounds of formula (I) according to the invention are administered orally once or twice or three times a day. According to a further embodiment, the compounds of formula (I) according to the invention are administered orally once or twice a day. According to a further embodiment, the compounds of formula (I) according to the invention are administered orally once a day. For the oral administration, a rapid release or a modified release dosage form may be used.

Unless stated otherwise, the percentages in the tests and examples which follow are percentages by weight; parts are parts by weight. Solvent ratios, dilution ratios and concentration data for the liquid/liquid solutions are based in each case on volume. “w/v” means “weight/volume”. For example, “10% w/v” means: 100 ml of solution or suspension comprise 10 g of substance.

### SPECIFIC EMBODIMENTS OF THE INVENTION

1. A compound for use in the treatment and/or prophylaxis of heart failure with preserved ejection fraction (HFpEF) in women selected from the group consisting of

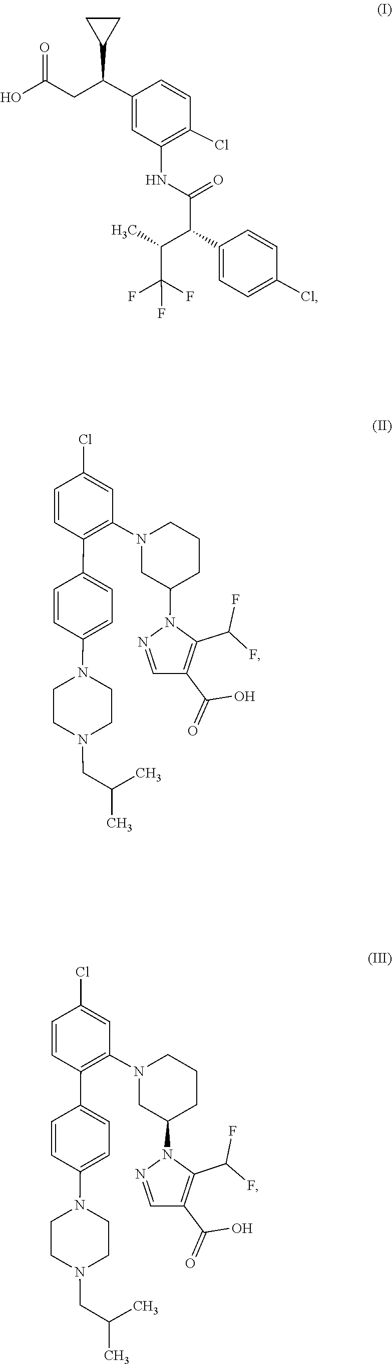 [SMILES: C[C@H]([C@H](C(=O)Nc1cc([C@@H](CC(=O)O)C2CC2)ccc1Cl)c1ccc(Cl)cc1)C(F)(F)F ; CC(C)CN1CCN(c2ccc(-c3ccc(Cl)cc3N3CCCC(n4ncc(C(=O)O)c4C(F)F)C3)cc2)CC1 ; CC(C)CN1CCN(c2ccc(-c3ccc(Cl)cc3N3CCC[C@@H](n4ncc(C(=O)O)c4C(F)F)C3)cc2)CC1]

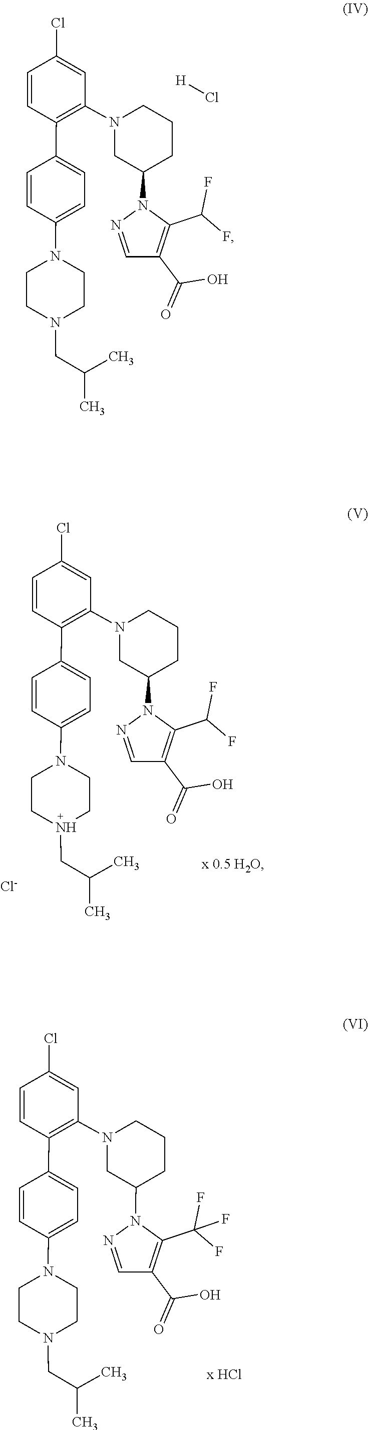

or one of the salts thereof, solvates thereof or solvates of the salts thereof.

2. A compound for use in the treatment and/or prophylaxis of heart failure with preserved ejection fraction (HFpEF) in women of the formula (I)

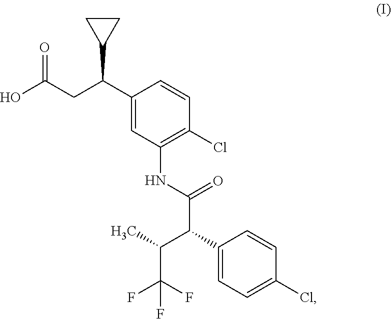 [SMILES: C[C@H]([C@H](C(=O)Nc1cc([C@@H](CC(=O)O)C2CC2)ccc1Cl)c1ccc(Cl)cc1)C(F)(F)F]

or one of the salts thereof, solvates thereof or solvates of the salts thereof.

3. A compound for use in the treatment and/or prophylaxis of heart failure with preserved ejection fraction (HFpEF) in women of the formula (II)

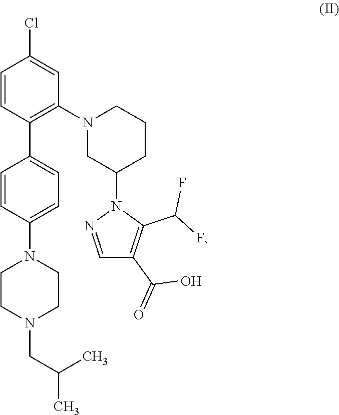 [SMILES: CC(C)CN1CCN(c2ccc(-c3ccc(Cl)cc3N3CCCC(n4ncc(C(=O)O)c4C(F)F)C3)cc2)CC1]

or one of the salts thereof, solvates thereof or solvates of the salts thereof.

4. A compound for use in the treatment and/or prophylaxis of heart failure with preserved ejection fraction (HFpEF) in women of the formula (III)

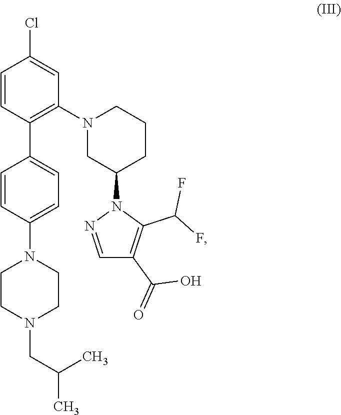 [SMILES: CC(C)CN1CCN(c2ccc(-c3ccc(Cl)cc3N3CCC[C@@H](n4ncc(C(=O)O)c4C(F)F)C3)cc2)CC1]

or one of the salts thereof, solvates thereof or solvates of the salts thereof.

5. A compound for use in the treatment and/or prophylaxis of heart failure with preserved ejection fraction (HFpEF) in women of the formula (IV)

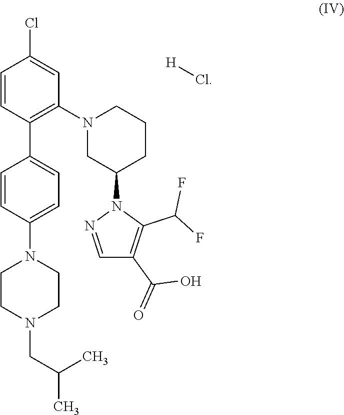 [SMILES: CC(C)CN1CCN(c2ccc(-c3ccc(Cl)cc3N3CCC[C@@H](n4ncc(C(=O)O)c4C(F)F)C3)cc2)CC1 ; Cl]

6. A compound for use in the treatment and/or prophylaxis of heart failure with preserved ejection fraction (HFpEF) in women of the formula (V)

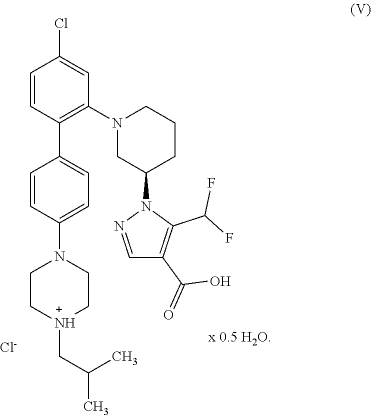 [SMILES: [Cl-] ; CC(C)C[NH+]1CCN(c2ccc(-c3ccc(Cl)cc3N3CCC[C@@H](n4ncc(C(=O)O)c4C(F)F)C3)cc2)CC1]

7. A compound for use in the treatment and/or prophylaxis of heart failure with preserved ejection fraction (HFpEF) in women of the formula (VI)

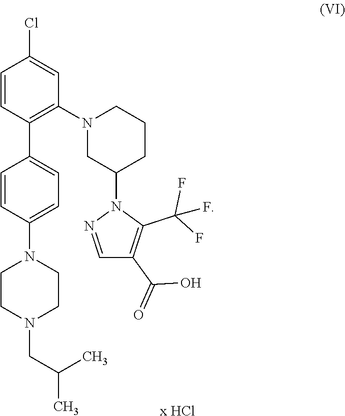 [SMILES: CC(C)CN1CCN(c2ccc(-c3ccc(Cl)cc3N3CCCC(n4ncc(C(=O)O)c4C(F)(F)F)C3)cc2)CC1 ; "x HCl"]

8. Use of a compound according to any one of claims 1 to 7 for producing a medicament for use in the treatment and/or prophylaxis of heart failure with preserved ejection fraction (HFpEF) in women.

9. Medicament comprising a compound according to any one of claims 1 to 7 in combination with an inert, nontoxic, pharmaceutically suitable excipient for use in the treatment and/or prophylaxis of heart failure with preserved ejection fraction (HFpEF) in women.

10. Medicament according to claim 9 comprising 0.1 mg to 500 mg, preferably 1 mg to 140 mg, more preferably 2.5 mg to 120 mg, also more preferably 20 mg to 120 mg, also more preferably 30 mg to 120 mg, also more preferably 2.5 mg to 50 mg, more preferably 2 mg to 50 mg, more preferably 2 mg to 40 mg, more preferably 2 mg to 30 mg, more preferably 2.5 mg to 40 mg, more preferably 2.5 mg to 30 mg of active ingredient.

11. Medicament according to claim 10 comprising 0.1 mg to 500 mg, preferably 1 mg to 140 mg, more preferably 2.5 mg to 120 mg, also more preferably 20 mg to 120 mg, also more preferably 30 mg to 120 mg, of compound of formula (I)

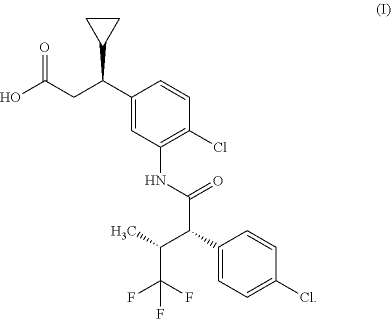 [SMILES: C[C@H]([C@H](C(=O)Nc1cc([C@@H](CC(=O)O)C2CC2)ccc1Cl)c1ccc(Cl)cc1)C(F)(F)F]

12. Medicament according to claim 10 comprising 0.1 mg to 500 mg, preferably 1 mg to 140 mg, more preferably also more preferably 2.5 mg to 50 mg, more preferably 2 mg to 50 mg, more preferably 2 mg to 40 mg, more preferably 2 mg to 30 mg, more preferably 2.5 mg to 40 mg, more preferably 2.5 mg to 30 mg of compound of formula (II)

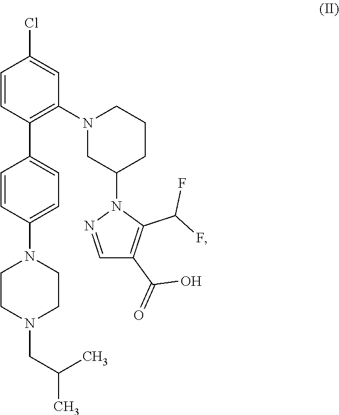 [SMILES: CC(C)CN1CCN(c2ccc(-c3ccc(Cl)cc3N3CCCC(n4ncc(C(=O)O)c4C(F)F)C3)cc2)CC1]

or one of the salts thereof, solvates thereof or solvates of the salts thereof.

13. Medicament according to claim 10 comprising 0.1 mg to 500 mg, preferably 1 mg to 140 mg, more preferably also more preferably 2.5 mg to 50 mg, more preferably 2 mg to 50 mg, more preferably 2 to 40 mg, more preferably 2 mg to 30 mg, more preferably 2.5 mg to 40 mg, more preferably 2.5 mg to 30 mg of compound of formula (111)

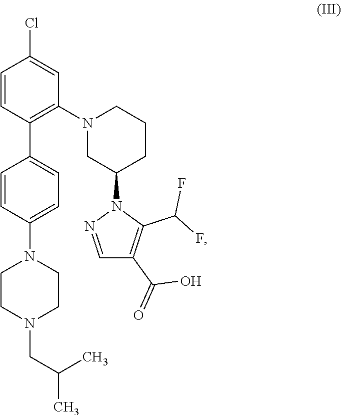 [SMILES: CC(C)CN1CCN(c2ccc(-c3ccc(Cl)cc3N3CCC[C@@H](n4ncc(C(=O)O)c4C(F)F)C3)cc2)CC1]

or one of the salts thereof, solvates thereof or solvates of the salts thereof.

14. Medicament according to claim 10 comprising 0.1 to 500 mg, preferably 1 mg to 140 mg, more preferably also more preferably 2.5 mg to 50 mg, more preferably 2 to 50 mg, more preferably 2 to 40 mg, more preferably 2 mg to 30 mg, more preferably 2.5 mg to 40 mg, more preferably 2.5 mg to 30 mg of compound of formula (IV)

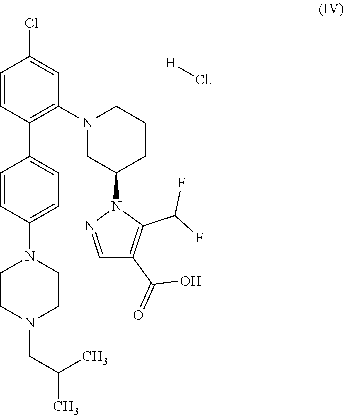 [SMILES: CC(C)CN1CCN(c2ccc(-c3ccc(Cl)cc3N3CCC[C@@H](n4ncc(C(=O)O)c4C(F)F)C3)cc2)CC1 ; Cl]

15. Medicament according to claim 10 comprising 0.1 to 500 mg, preferably 1 mg to 140 mg, more preferably also more preferably 2.5 mg to 50 mg, more preferably 2 to 50 mg, more preferably 2 to 40 mg, more preferably 2 mg to 30 mg, more preferably 2.5 mg to 40 mg, more preferably 2.5 mg to 30 mg of compound of formula (V)

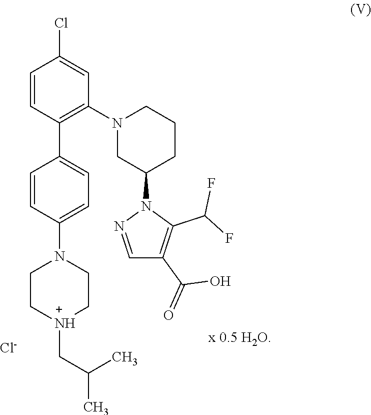 [SMILES: [Cl-] ; CC(C)C[NH+]1CCN(c2ccc(-c3ccc(Cl)cc3N3CCC[C@@H](n4ncc(C(=O)O)c4C(F)F)C3)cc2)CC1]

16. Medicament according to claim 10 comprising 0.1 to 500 mg, preferably 1 mg to 140 mg, more preferably also more preferably 2.5 mg to 50 mg, more preferably 2 to 50 mg, more preferably 2 to 40 mg, more preferably 2 mg to 30 mg, more preferably 2.5 mg to 40 mg, more preferably 2.5 mg to 30 mg of compound of formula (VI)

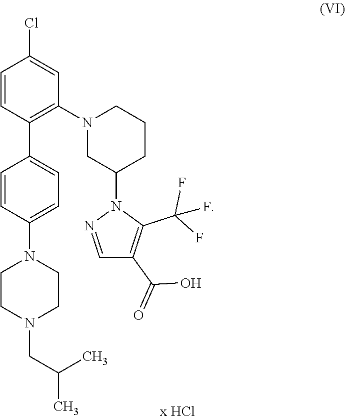 [SMILES: CC(C)CN1CCN(c2ccc(-c3ccc(Cl)cc3N3CCCC(n4ncc(C(=O)O)c4C(F)(F)F)C3)cc2)CC1 ; "x HCl"]

17. Method for the treatment and/or prophylaxis of heart failure with preserved ejection fraction (HFpEF) in women by administration of a therapeutically effective amount of at least one compound according to any one of claims 1 to 7 or of a medicament according to any one of claims 9 to 16.

### EXPERIMENTAL SECTION

### 1. A. Chemical Test Compounds

### Example 1

(3S)-3-(4-chloro-3-{[(2S,3R)-2-(4-chlorophenyl)-4,4,4-trifluoro-3-methylbutanoyl]amino}phenyl)-3-cyclopropylpropanoic acid (runcaciguat) of the formula (I)

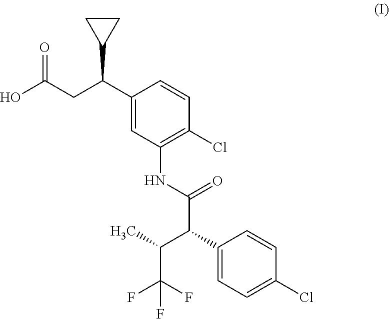 [SMILES: C[C@H]([C@H](C(=O)Nc1cc([C@@H](CC(=O)O)C2CC2)ccc1Cl)c1ccc(Cl)cc1)C(F)(F)F]

was synthesized as disclosed in WO2012/139888.

### Example 2

1-[1-{4-Chloro-4′-[4-(2-methylpropyl)piperazin-1-yl][1,1′-biphenyl]-2-yl}piperidin-3-yl]-5-(difluoromethyl)-1H-pyrazole-4-carboxylic acid of the formula (II)

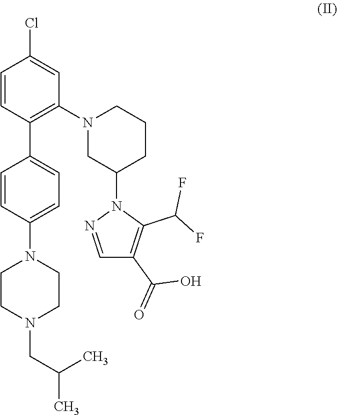 [SMILES: CC(C)CN1CCN(c2ccc(-c3ccc(Cl)cc3N3CCCC(n4ncc(C(=O)O)c4C(F)F)C3)cc2)CC1]

was synthesized as disclosed in WO2022/122910.

### Example 3

1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid of the formula (III)

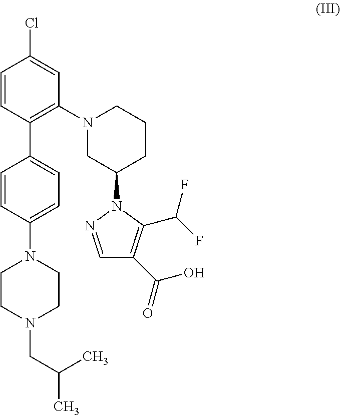 [SMILES: CC(C)CN1CCN(c2ccc(-c3ccc(Cl)cc3N3CCC[C@@H](n4ncc(C(=O)O)c4C(F)F)C3)cc2)CC1]

was synthesized as disclosed in WO2022/122910.

### EXAMPLE 4

1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride of the formula (IV)

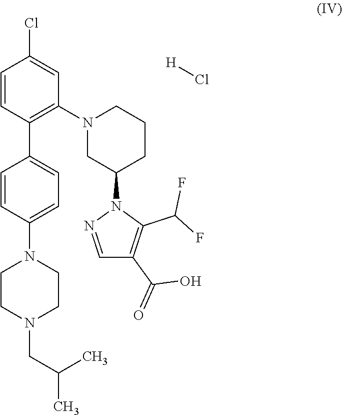 [SMILES: CC(C)CN1CCN(c2ccc(-c3ccc(Cl)cc3N3CCC[C@@H](n4ncc(C(=O)O)c4C(F)F)C3)cc2)CC1 ; Cl]

was synthesized as disclosed in WO2022/122910.

### Example 5

1-{3(R)-1-[4-Chloro-4′-(4-isobutylpiperazin-1-yl)[biphenyl]-2-yl]piperidin-3-yl}-5-(difluoromethyl)-1H-pyrazole-4-carboxylic acid hydrochloride hemihydrate of the formula (V)

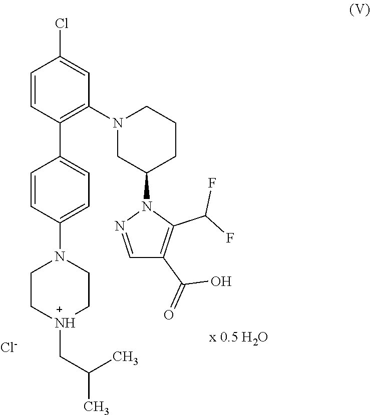 [SMILES: [Cl-] ; CC(C)C[NH+]1CCN(c2ccc(-c3ccc(Cl)cc3N3CCC[C@@H](n4ncc(C(=O)O)c4C(F)F)C3)cc2)CC1]

was synthesized as disclosed in WO2022/122910.

### Example 6

1-[1-{4-chloro-4′-[4-(2-methylpropyl)piperazin-1-yl][1,1′-biphenyl]-2-yl}piperidin-3-yl]-5-(trifluoromethyl)-1H-pyrazole-4-carboxylic acid hydrochloride (Enantiomer 1) of the formula (VI)

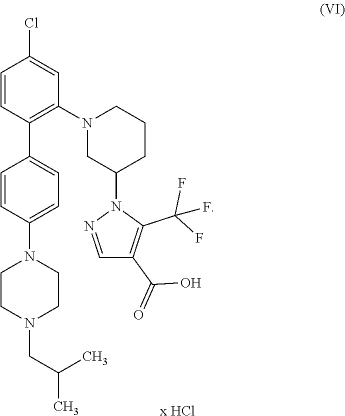 [SMILES: CC(C)CN1CCN(c2ccc(-c3ccc(Cl)cc3N3CCCC(n4ncc(C(=O)O)c4C(F)(F)F)C3)cc2)CC1 ; "x HCl"]

was obtained as disclosed in WO2022/122914.

### B. Assessment of Pharmacological Efficacy and Pharmacokinetic Profile

The following abbreviations are used:

| | |
|---|---|
| ATP | adenosine triphosphate |
| BID | Bis in die, twice a day |
| BSA | bovine serum albumin: |
| DTT | dithiothreitol |
| OD | once daily |
| p.o. | per |
| os | |

### Biological Investigations

The example testing experiments described herein serve to illustrate the present invention and the invention is not limited to the examples given.

The following assays can be used to illustrate the commercial utility of the compounds according to the present invention.

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values calculated utilizing data sets obtained from testing of one or more synthetic batch.

The in vitro activity of the compounds of the present invention can be demonstrated in the following assays.

The pharmacological action of the compounds of the invention can be demonstrated in the following assays:

### B-1. RenTG Rat Model

### Ethical Statement:

All animal experiments are done in accordance to the current national legislation (German protection of animals act and the EU directives on the protection of animals used for scientific purposes). All performed studies were approved by the regional regulatory authority (LANUV NRW in Germany) and by the institutional animal care and use committee of Bayer AG.

### Study Design and Read Outs:

Male and female rennin-transgenic rats [TG(mRRen2)27] (RenTG rats) at the age of 8 weeks were used. Rats were randomized in groups with 12-24 rats/group. All groups were then chronically supplemented with L-NAME administrated via drinking water (30-50 mg/l). The L-NAME was dissolved in tap water and stored by room temperature. All groups were treated via oral gavage with either placebo/vehicle consisting of 10% transcutol, 20% cremophor and 70% tap water or different doses of the respective verum. The verum was dissolved in the vehicle and prepared freshly before administration. Study duration with concomitant L-NAME administration and treatment was up to 12 weeks. The baseline blood pressure was measured via tail-cuff method before the start of the treatment and after different time points during the study. Mortality and body weight were assessed on a daily basis. Urine samples were collected via the use of metabolic cages and urine was collected over a period of six hours to quantify proteinuria as correlate of kidney function and different urinary biomarkers for kidney and organ damage. At the end of the study all animals were kept in depth narcotic and bled via aortic puncture and sacrificed by permanent inhaled isoflurane, blood was taken in order to assess plasma parameters, and heart and kidney were weighed and harvested for histopathological evaluation.

### B-1.1 Survival Rate Assessment in Male Rats

The survival rate in % of the RenTG/L-NAME-supplemented male rats treated with either placebo, or 1, 3 and 10 mg/kg runcaciguat (example 1) BID was investigated (see table 1).

TABLE 1
survival rate in % of the RenTG/L-NAME-supplemented male rats

| | n start | n final | % survival |
|---|---|---|---|
| placebo | 24 | 10 | 42 |
| example 1, 1 mg/kg | 18 | 8 | 44 |
| example 1, 3 mg/kg | 18 | 11 | 61 |
| example 1, 10 mg/kg | 18 | 13 | 72 |

Example 1 (runcaciguat) was effective in the RenTG rat model and dose-dependently increased the survival rates (see FIG. 2). In the placebo-treated control group 58% of rats died. In contrast, the p.o. BID treatment with example 1 (runcaciguat) at 1 mg/kg, 3 mg/kg and 10 mg/kg, reduced mortality to 56%, 39% and 28%, respectively. Whereas the 3 mg/kg example 1 (runcaciguat) treatment showed a strong numerical trend, the 10 mg/kg treatment arm reached statistical significance. In summary, example 1 (runcaciguat) treatment decreased mortality rates in a dose dependent manner.

To further confirm the treatment effect of example 1 (runcaciguat) in the RenTG rats we also assessed kidney function by analysis of proteinuria (urinary protein to creatine ratio, uPCR).

B-1.2 Kidney Function Assessment by Analysis of Proteinuria (Urinary Protein to Creatine Ratio, uPCR) in Male Rats

Urinary protein to creatinine ratio in the RenTG/L-NAME-supplemented male rats treated with either placebo (set as 100%) or with 1, 3 and 10 mg/kg example 1 (runcaciguat) BID was investigated (see table is 2).

TABLE 2
Urinary protein to creatinine ratio in
the RenTG/L-NAME-supplemented male rats

| | uPCR % (normalized to placebo) |
|---|---|
| placebo | 100 |
| example 1, 1 mg/kg | 85.4 |
| example 1, 3 mg/kg | 71.1 |
| example 1, 10 mg/kg | 60.6 |

Example 1 (runcaciguat) led to a dose dependent reduction of proteinuria in the RenTG rats of 15%, 29% and 39% in the 1, 3 and 10 mg/kg BID treatment group, respectively, whereby the effects in the 3 and 10 mg/kg BID treatment arms were statistically significant (see FIG. 3).

In summary, example 1 (runcaciguat) was dose-dependently effective in the male RenTG rat model and improved both survival and kidney function.

However, the effects of example 1 (runcaciguat) in female RenTG rats were not known.

For further evaluation of runcaciguat in female RenTG rats we choose the example 1 (runcaciguat) dose of 3 mg/kg BID which was effective in male rats. In addition, the same dose of example 3 was used. As in male rats, survival rates and kidney function were investigated.

### B-1.3 Survival Rate Assessment in Female Rats

The survival rate in % of the RenTG/L-NAME-supplemented female rats treated with either placebo, or 3 mg/kg example 1 (runcaciguat) or 3 mg/kg example 3 BID was investigated (see table 3).

TABLE 3
survival rate in % of the RenTG/L-
NAME-supplemented female rats

| | n start | n final | % survival |
|---|---|---|---|
| healthy control | 8 | 8 | 100 |
| placebo | 28 | 18 | 64.3 |
| example 1 | 13 | 13 | 100 |
| example 3 | 13 | 12 | 92.3 |

It turned out, that the effects of example 1 (runcaciguat) and example 3 were significantly higher in the female animals in which all animals (100%) treated with 3 mg/kg example 1 (runcaciguat) survived (see FIG. 4) in comparisons to only 60% in the male groups (see B 1.1, FIG. 2). Also the treatment with example 3 resulted in a substantial survival benefit and more than 90% of female RenTG rats treated with example 3 survived.

Since mortality in the female studies tends to be lower (64% of placebo-treated animal in the female control group survived compared to 42% in the male control group), we also analyzed mortality in male rats when 64% of the male control animals were still alive. At this point the survival rates for male rats treated with example 1 (runcacgiguat) at 3 mg/kg BID were 70%. Again, this is a 30% lower survival compared to female rats treated with example 1 (runcaciguat) 3 mg/kg BID.

To further substantiate our findings, we analyzed proteinuria in female RenTG rats treated with example 1 (runcaciguat) and example 3.

B-1.4. Kidney Function Assessment by Analysis of Proteinuria (Urinary Protein to Creatine Ratio, uPCR) in Female Rats

Urinary protein to creatinine ratio in the RenTG/L-NAME-supplemented female rats treated with either placebo (set as 100%) or 3 mg/kg example 1 BID or example 3 BID respectively. Data are mean±SEM.

TABLE 4
Urinary protein to creatinine ratio in the
RenTG/L-NAME-supplemented female rats

| | uPCR % (normalized to placebo) |
|---|---|
| healthy control | 3.5 |
| placebo | 100 |
| example 1 | 44.5 |
| example 3 | 16.4 |

3 mg/kg BID of either example 1 (runcaciguat) or example 3 were able to significantly reduce proteinuria by 55% and 84% respectively (see FIG. 5). This was again more effective compared to male rats in which 3 mg/kg BID example 1 (runcaciguat) treatment resulted in a 29% proteinuria reduction.

These data confirmed the mortality outcome in male versus female RenTG rats and sGC activators had greater effects in female compared to male RenTG rats.

In summary, these data strongly suggest that sGC activators, especially example 1 (runcaciguat) and example 3 might represent a novel and highly effective treatment option for HFpEF, but will become especially useful and effective for the treatment of female HFpEF patients.

The reason for this significantly higher benefit in female HFpEF rats is not understood and was novel for us. This has not been studied to our knowledge so far. However, one may speculate that an increased oxidative stress and endothelial dysfunction which have been described as important pathomechanisms in HFpEF (J Am Coll Cardiol 2020; 75(9):1074-1082, for review) could be responsible for that. The compound class of sGC activators can specifically bind to heme-free sGC and simulate cGMP synthesis in the absence of NO under conditions of oxidative stress (see FIG. 2). Interestingly, women with HFpEF are mostly post-menopausal and have a variety of comorbidities like diabetes, kidney disease, hypertension, and obesity which can increase oxidative stress. Therefore, treating female HFpEF patients with sGC activators could become a very effective treatment option and should be further investigated in the future and substantiated by respective clinical studies.

In B-1.3 survival assessment for Example 3 (compound of formula (II)-(VI)) was included in female rats. These results imply a very high efficacy of the sGC activator example 3 on survival rate (92,3%) in female rats but did not directly show the male versus female comparison like for example 1.

### B-1.5. Survival Rate Assessment in Male, Female, and Female Ovariectomized (OVX) Rats

Head-to-head comparison of the sGC activator Example 3 in male rats, female rats and in female rats with ovariectomy (OVX) in a once daily dosing (OD) regimen showed that the survival rate in male rats was 7% lower than in female rats. In male rat 93% of rats survived whereas in female rats all the rats survived in the sGC activator treatment arm (100%). There was no difference in survival rates in female OVX rats compared to non-OVX rats.

### Tables 5a-c: Survival Rate in % of the RenTG/L-NAME-Supplemented Male (a), Female (b) and Female OVX (c) Rats

TABLE 5a
male rats

| | n start | n final | % survival |
|---|---|---|---|
| placebo | 15 | 9 | 60 |
| Example 3, 3 mg/kg | 15 | 14 | 93 |
| OD | | | |

TABLE 5b
female rats

| | n start | n final | % survival |
|---|---|---|---|
| placebo | 15 | 7 | 47 |
| Example 3, 3 mg/kg | 15 | 15 | 100 |
| OD | | | |

TABLE 5c
female OVX rats

| | n start | n final | % survival |
|---|---|---|---|
| placebo | 15 | 6 | 40 |
| Example 3, 3 mg/kg | 15 | 15 | 100 |
| OD | | | |

B-1.6. Kidney Function Assessment by Analysis of Proteinuria (Urinary Protein to Creatine Ratio, uPCR) in Male, Female, and Female Ovariectomized (OVX) Rats

The most striking differences between male and female were seen on proteinuria. In male rats, proteinuria was significantly reduced by 57% when treated with the sGC activator Example 3. In female rats these effects were much more pronounced, and proteinuria was reduced by 88% and by 99% in female rats without OVX and with OVX, respectively. These data clearly show the superiority of the sGC activator example 3 in female rats compared to male rats and further reduced in OVX rats. These data strongly suggest that female postmenopausal patients could profit most from treatment with an sGC activator.

TABLE 6
Urinary protein to creatinine ratio in the RenTG/L-
NAME-supplemented male, female and female OVX rats

| | male | female | female OVX |
|---|---|---|---|
| placebo | 100 | 100 | 100 |
| example 3, 3 mg/kg OD | 43 | 12 | 1 |

### B-2. HFpEF Clinical Investigation

Women suffering from HFpEF [defined as LVEF≥45% plus structural heart changes such as left-ventricular hypertrophy or left-atrial enlargement and NT-pro BNP values 300 pg/mL (in the absence of atrial fibrillation) or 600 pg/mL (in the presence of atrial fibrillation) at randomization] are randomized within 3 months of heart failure decompensation defined as heart failure hospitalization or need for iv diuretic treatment for heart failure without hospitalization to treatment with either placebo or different doses of the sgc activator example 1 or example 3 in a double-blinded multicenter study. Primary readout parameter is the change in 6 minute walking distance (6MWD) from 24 weeks of treatment to baseline, secondary readout parameters are safety and tolerability as measured by the numbers of treatment-emergent adverse events. Women who are not able to participate in an aerobic exercise test due to exercise limiting comorbidities such as e.g. chronic obstructive pulmonary disease, interstitial lung disease, intermittent claudication, orthopedic or neurological conditions interfering with exercise capacity, anemia, use of wheel chairs, walking aids or nasal oxygen are excluded from the study. To detect a true Delta of at least 30 m increase in 6 MWD in the sgc activator group compared to the placebo group and assuming a common standard deviation of 70 meter (alpha of 5% and 90% power), 116 patients are required per arm in order to get a p-value less than 0.05.

### C. Working Examples of Pharmaceutical Compositions

Modified release (GITS) formulations comprising example 1 are disclosed in WO 2020/020789.

The compounds of the invention can be converted to pharmaceutical preparations as follows:

### Tablet:

### Composition:

100 mg of the compound according to the invention, 50 mg of lactose (monohydrate), 50 mg of corn starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm.

### Production:

The mixture of compound of the invention, lactose and starch is granulated with a 5% solution (w/w) of the PVP in water. The granules are dried and then mixed with the magnesium stearate for 5 minutes. This mixture is compressed using a conventional tableting press (see above for format of the tablet). The guide value used for the pressing is a pressing force of 15 kN.

### Suspension for Oral Administration:

### Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

### Production:

The Rhodigel is suspended in ethanol; the compound of the invention is added to the suspension. The water is added while stirring. The mixture is stirred for about 6 h until the swelling of the Rhodigel is complete.

### Solution for Oral Administration:

### Composition:

500 mg of the compound of the invention, 2.5 g of polysorbate and 97 g of polyethylene glycol 400. 20 g of oral solution correspond to a single dose of 100 mg of the compound of the invention.

### Production:

The compound of the invention is suspended in the mixture of polyethylene glycol and polysorbate with stirring. The stirring process is continued until the compound according to the invention has completely dissolved.

i.v. Solution:

The compound according to the invention is dissolved in a concentration below the saturation solubility in a physiologically tolerated solvent (e.g. isotonic saline, 5% glucose solution and/or 30% PEG 400 solution). The solution is sterilized by filtration and used to fill sterile and pyrogen-free injection containers.

### FIGURES

FIG. 1: Mode of action of sGC stimulators and sGC activators

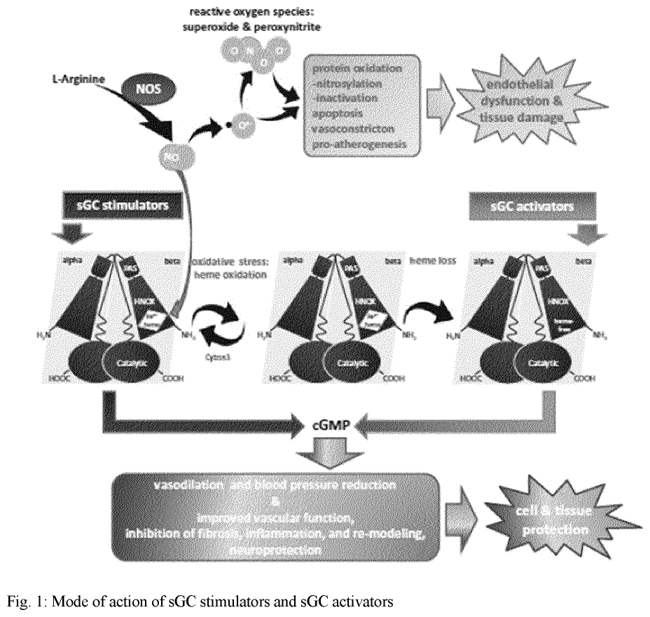

FIG. 2: Survival in % of RenTG and L-NAME-supplemented male rats with either placebo, or 1, 3 and 10 mg/kg runcaciguat (example 1) BID.

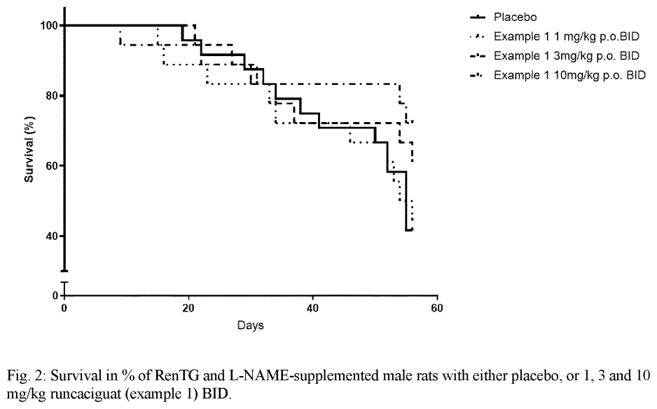

FIG. 3: Urinary protein to creatinine ratio in male rats treated with either placebo or different doses of is example 1, i.e. 1, 3 and 10 mg/kg, BID. Data are mean±SEM

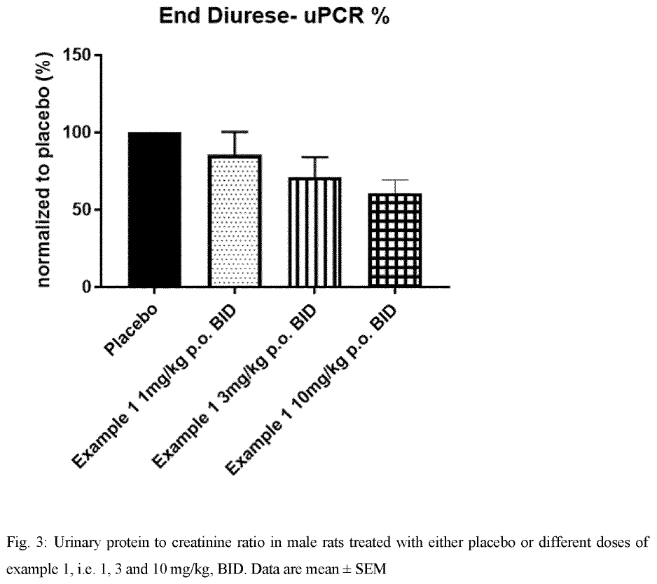

FIG. 4: Survival in % of RenTG and L-NAME-supplemented female rats treated with either placebo, or 3 mg/kg example 1 (runcaciguat) or 3 mg/kg example 3 BID

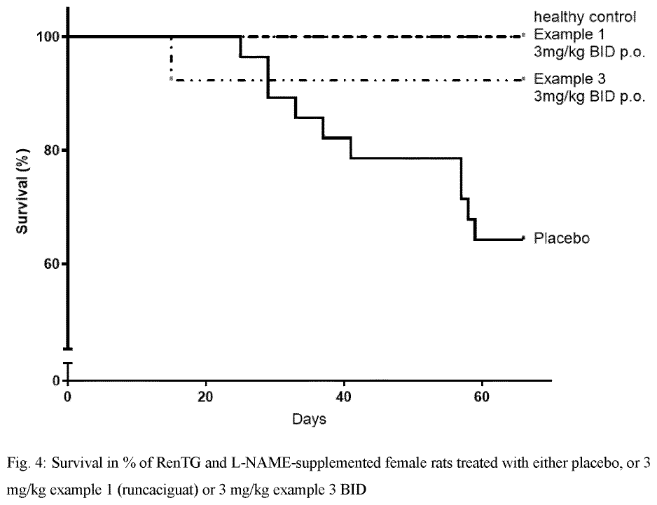

FIG. 5: Urinary protein to creatinine ratio in female rats treated with either placebo (set as 100%) or 3 mg/kg example 1 BID or example 3 BID, respectively. Data are mean±SEM.

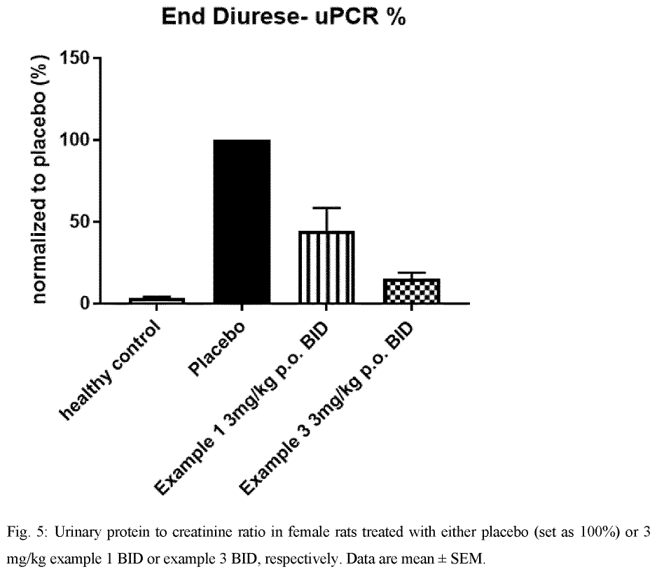

1. A compound for use in the treatment and/or prophylaxis of heart failure with preserved ejection fraction (HFpEF) in women selected from the group consisting of 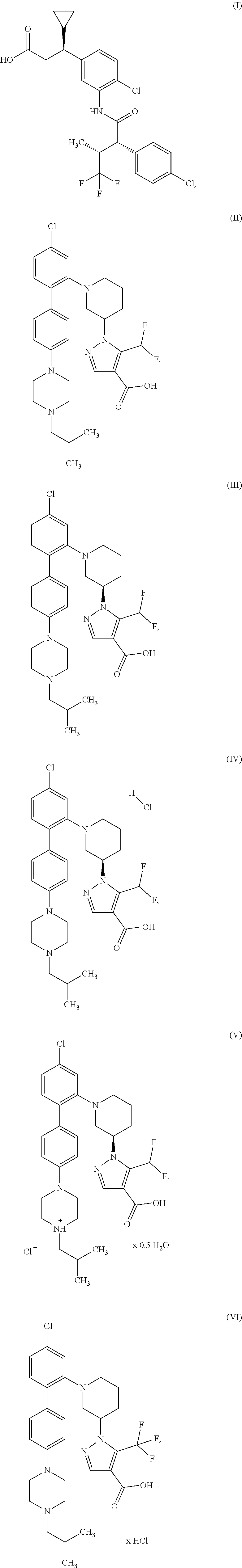
and one of the salts thereof, solvates thereof or solvates of the salts thereof.

2. A compound for use in the treatment and/or prophylaxis of heart failure with preserved ejection fraction (HFpEF) in women according to claim 1 of the formula (I) 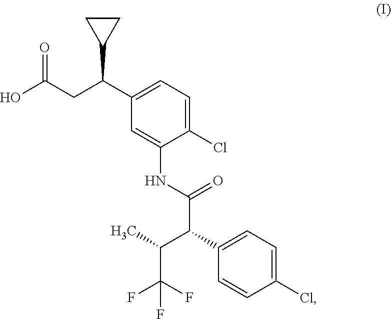 [SMILES: C[C@H]([C@H](C(=O)Nc1cc([C@@H](CC(=O)O)C2CC2)ccc1Cl)c1ccc(Cl)cc1)C(F)(F)F]
or one of the salts thereof, solvates thereof or solvates of the salts thereof.

3. A compound for use in the treatment and/or prophylaxis of heart failure with preserved ejection fraction (HFpEF) in women according to claim 1 of the formula (II) 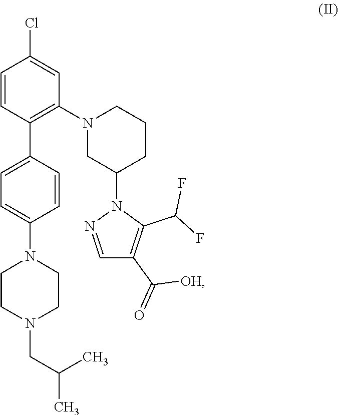 [SMILES: CC(C)CN1CCN(c2ccc(-c3ccc(Cl)cc3N3CCCC(n4ncc(C(=O)O)c4C(F)F)C3)cc2)CC1]
or one of the salts thereof, solvates thereof or solvates of the salts thereof.

4. A compound for use in the treatment and/or prophylaxis of heart failure with preserved ejection fraction (HFpEF) in women according to claim 1 of the formula (III) 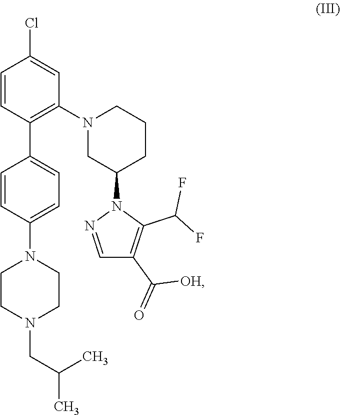 [SMILES: CC(C)CN1CCN(c2ccc(-c3ccc(Cl)cc3N3CCC[C@@H](n4ncc(C(=O)O)c4C(F)F)C3)cc2)CC1]
or one of the salts thereof, solvates thereof or solvates of the salts thereof.

5. A compound for use in the treatment and/or prophylaxis of heart failure with preserved ejection fraction (HFpEF) in women according to claim 1 of the formula (IV)

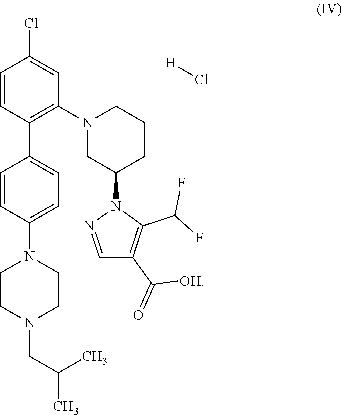 [SMILES: CC(C)CN1CCN(c2ccc(-c3ccc(Cl)cc3N3CCC[C@@H](n4ncc(C(=O)O)c4C(F)F)C3)cc2)CC1 ; Cl]

6. A compound for use in the treatment and/or prophylaxis of heart failure with preserved ejection fraction (HFpEF) in women according to claim 1 of the formula (V)

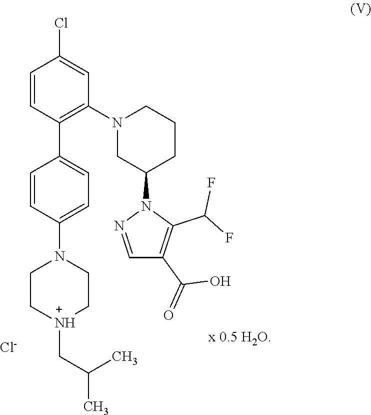 [SMILES: [Cl-] ; CC(C)C[NH+]1CCN(c2ccc(-c3ccc(Cl)cc3N3CCC[C@@H](n4ncc(C(=O)O)c4C(F)F)C3)cc2)CC1]

7. A compound for use in the treatment and/or prophylaxis of heart failure with preserved ejection fraction (HFpEF) in women according to claim 1 of the formula (VI)

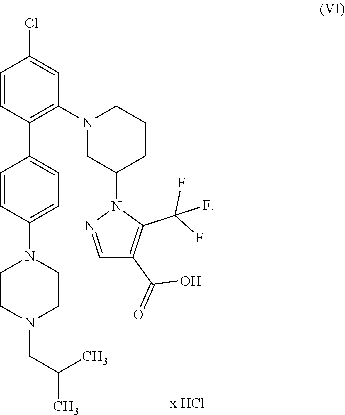 [SMILES: CC(C)CN1CCN(c2ccc(-c3ccc(Cl)cc3N3CCCC(n4ncc(C(=O)O)c4C(F)(F)F)C3)cc2)CC1]

8. Medicament comprising a compound according to claim 1 in combination with an inert, nontoxic, pharmaceutically suitable excipient for use in the treatment and/or prophylaxis of heart failure with preserved ejection fraction (HFpEF) in women.

9. Medicament according to claim 8 comprising 0.1 mg to 500 mg, preferably 1 mg to 140 mg, more preferably 2.5 mg to 120 mg, also more preferably 20 mg to 120 mg, also more preferably 30 mg to 120 mg, also more preferably 2.5 mg to 50 mg, more preferably 2 mg to 50 mg, more preferably 2 mg to 40 mg, more preferably 2 mg to 30 mg, more preferably 2.5 mg to 40 mg, more preferably 2.5 mg to 30 mg of the compound.

10. Medicament according to claim 9 comprising 0.1 mg to 500 mg, preferably 1 mg to 140 mg, more preferably 2.5 mg to 120 mg, also more preferably 20 mg to 120 mg, also more preferably 30 mg to 120 mg, of a compound of formula (I)

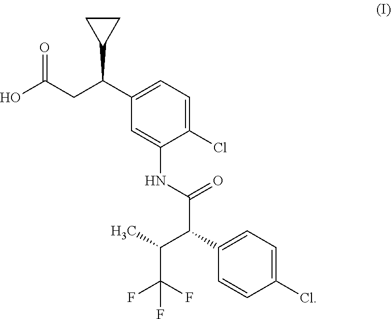 [SMILES: C[C@H]([C@H](C(=O)Nc1cc([C@@H](CC(=O)O)C2CC2)ccc1Cl)c1ccc(Cl)cc1)C(F)(F)F]

11. Medicament according to claim 9 comprising 0.1 mg to 500 mg, preferably 1 mg to 140 mg, more preferably also more preferably 2.5 mg to 50 mg, more preferably 2 mg to 50 mg, more preferably 2 mg to 40 mg, more preferably 2 mg to 30 mg, more preferably 2.5 mg to 40 mg, more preferably 2.5 mg to 30 mg of compound of formula (II) 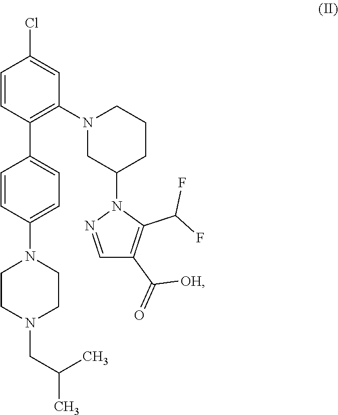 [SMILES: CC(C)CN1CCN(c2ccc(-c3ccc(Cl)cc3N3CCCC(n4ncc(C(=O)O)c4C(F)F)C3)cc2)CC1]
or one of the salts thereof, solvates thereof or solvates of the salts thereof.

12. Medicament according to claim 9 comprising 0.1 mg to 500 mg, preferably 1 mg to 140 mg, more preferably also more preferably 2.5 mg to 50 mg, more preferably 2 mg to 50 mg, more preferably 2 to 40 mg, more preferably 2 mg to 30 mg, more preferably 2.5 mg to 40 mg, more preferably 2.5 mg to 30 mg of compound of formula (III) 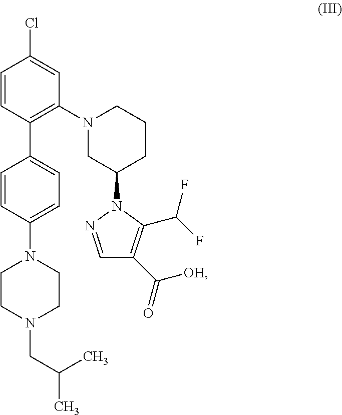 [SMILES: CC(C)CN1CCN(c2ccc(-c3ccc(Cl)cc3N3CCC[C@@H](n4ncc(C(=O)O)c4C(F)F)C3)cc2)CC1]
or one of the salts thereof, solvates thereof or solvates of the salts thereof.

13. Medicament according to claim 9 comprising 0.1 to 500 mg, preferably 1 mg to 140 mg, more preferably also more preferably 2.5 mg to 50 mg, more preferably 2 to 50 mg, more preferably 2 to 40 mg, more preferably 2 mg to 30 mg, more preferably 2.5 mg to 40 mg, more preferably 2.5 mg to 30 mg of compound of formula (IV)

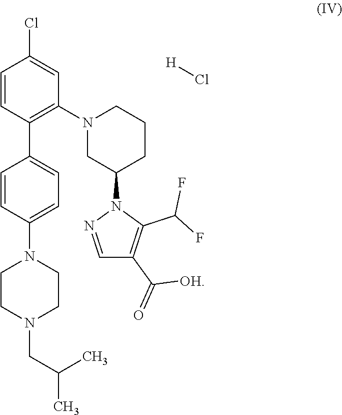 [SMILES: CC(C)CN1CCN(c2ccc(-c3ccc(Cl)cc3N3CCC[C@@H](n4ncc(C(=O)O)c4C(F)F)C3)cc2)CC1 ; Cl]

14. Medicament according to claim 9 comprising 0.1 to 500 mg, preferably 1 mg to 140 mg, more preferably also more preferably 2.5 mg to 50 mg, more preferably 2 to 50 mg, more preferably 2 to 40 mg, more preferably 2 mg to 30 mg, more preferably 2.5 mg to 40 mg, more preferably 2.5 mg to 30 mg of compound of formula (VI)

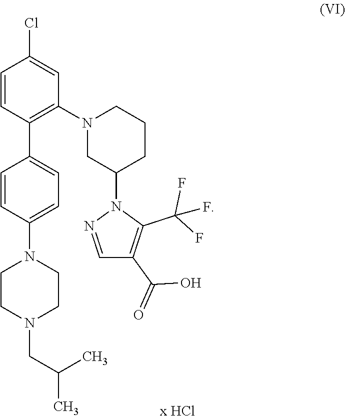 [SMILES: CC(C)CN1CCN(c2ccc(-c3ccc(Cl)cc3N3CCCC(n4ncc(C(=O)O)c4C(F)(F)F)C3)cc2)CC1 ; "x HCl"]

15. A method for the treatment of heart failure with preserved ejection fraction (HFpEF) in women by administration of a therapeutically effective amount of at least one compound according to claim 1.
